(19) United States
(12) Patent Application Publication
(10) Pub. No.: US 2016/0100778 A1
(43) Pub. Date: Apr. 14, 2016

(54) BIOSENSOR AND WEARABLE DEVICE FOR DETECTING BIOINFORMATION INCLUDING HYBRID ELECTRONIC SHEET

(71) Applicant: KOREA INSTITUTE OF SCIENCE AND TECHNOLOGY, Seoul (KR)

(72) Inventors: Hyunjung YI, Seoul (KR); Ki Young LEE, Seoul (KR); Seungwoo LEE, Seoul (KR); Cha Un JANG, Seoul (KR); Joonyeon CHANG, Seoul (KR)

(21) Appl. No.: 14/740,691

(22) Filed: Jun. 16, 2015

(30) Foreign Application Priority Data
Oct. 10, 2014 (KR) 10-2014-0136992
Mar. 11, 2015 (KR) 10-2015-0034037

(51) Int. Cl. A61B 5/1486; A61B 5/00; A61B 5/145

(52) U.S. Cl. CPC A61B 5/1486; A61B 5/14532; A61B 5/6821

(57) ABSTRACT

Provided are a biosensor and a wearable device for detecting bioinformation including a hybrid electronic sheet. The biosensor has high electrochemical activity, allows DET-based detection of an analyte in a sample, and has an electrode harmless to the human body to detect an analyte with high sensitivity and selectivity, thereby being usefully applied to a wearable device of detecting bioinformation.

### CROSS-REFERENCE TO RELATED APPLICATIONS

This application claims the benefit of Korean Patent Application No. 10-2014-0136992, filed on Oct. 10, 2014, and Korean Patent Application No. 10-2015-0034037, filed on Mar. 11, 2015, in the Korean Intellectual Property Office, the disclosures of which are incorporated herein in their entireties by reference.

### BACKGROUND

1. Field

The present disclosure relates to a biosensor and a wearable device for detecting bioinformation including a hybrid electronic sheet.

2. Description of the Related Art

With improvement of the quality of life and progress of medical technology, human life expectancy is rising, and with the growing number of older people, interest and investment are focused on self-diagnosis of health conditions.

In biosensor fields for self-diagnosis, a wearable biosensor is gaining interest, because it is not an invasive blood testing method, but a method of measuring glucose levels in the body fluid (saliva, tear, etc.) of a patient wearing the sensor to transmit the monitored information to an external device via wireless communication. Since the glucose levels in the body fluid such as tear, etc. are 10 to 20 times lower than the corresponding levels in the blood, it is required to develop a wearable biosensor which has higher sensitivity than a sensor of measuring the blood glucose levels in order to effectively detect such low glucose levels. Further, since the wearable biosensor is attached to the human body, its size must be greatly reduced using a patternable material and its properties in terms of flexibility, transparency, and surface adhesion must be improved.

To develop biosensors with improved properties, intensive effort has been focused on 3rd-generation biosensors of directly detecting changes in redox state of a reagent fixed on the surface of the sensor without mediators. Glucose sensors based on the 3rd-generation biosensor concept can efficiently exclude the interference of ascorbic acid (AA) and uric acid (UA) due to direct electron transfer (DET). Further, reaction of glucose oxidase with glucose is directly transferred to an electrode due to DET without any mediators, and thus there is improvement in terms of efficiency or accuracy of the sensor.

To fabricate such DET-based 3rd-generation biosensors, however, it is required to construct a sensor platform using nanomaterials by a complicated process. This fabrication process of the sensor requires much cost because reproducibility is low and it is difficult to control sensitivity of the sensor. Further, to realize the DET-based 3^{rd}-generation biosensor in a human body-attachable form, patterning of the nanomaterials is also required. Further, upon measurement, the sensor should be operated with not a reference electrode which may be dissolved into the body fluid but a reference electrode using a harmless electrode. Accordingly, there is a demand for a biosensor having improved sensitivity, of which a fabrication process requires low cost and patterning is possible.

### SUMMARY

An aspect provides a biosensor including a substrate; an electronic sheet formed on the substrate; and an analyte-binding material immobilized on the electronic sheet, in which the electronic sheet includes a graphitic material and a phage binding to the graphitic material, and the binding of the graphitic material and the phage occurs between a peptide displayed on a coat protein of the phage or a fragment thereof and the graphitic material.

Another aspect provides a wearable device for detecting bioinformation including the biosensor.

### BRIEF DESCRIPTION OF THE DRAWINGS

These and/or other aspects will become apparent and more readily appreciated from the following description of the embodiments, taken in conjunction with the accompanying drawings in which:

FIGS. 1A through 1D are schematic illustrations showing an electrode including a hybrid electronic sheet according to a specific embodiment;

FIGS. 2A through 2E are schematic illustrations showing the electrode including the hybrid electronic sheet on which an analyte-binding material is immobilized according to a specific embodiment;

FIG. 3 is a schematic illustration showing a biosensor according to a specific embodiment;

FIG. 4 is a cross-sectional view of the biosensor according to an exemplary embodiment along the Y-axis;

FIG. 5 is a cross-sectional view of the biosensor according to an exemplary embodiment along the X-axis;

FIG. 6 is a perspective view of a cover of the biosensor according to a specific embodiment;

FIG. 7 is a cross-sectional view of the biosensor according to a specific embodiment;

FIG. 8 is a perspective view of a cross-section of the cover of the biosensor according to a specific embodiment;

FIG. 9 is a schematic illustration showing a principle of DET reaction of the analyte-binding material on the hybrid electronic sheet according to a specific embodiment;

FIG. 10A is a schematic illustration showing a production process of the hybrid electronic sheet according to a specific embodiment;

FIG. 10B is a schematic illustration showing a formation principle of the hybrid electronic sheet according to a specific embodiment;

FIG. 10C is a graph showing concentration polarization in the formation principle of the hybrid electronic sheet according to a specific embodiment;

FIG. 11 is an image of a large-area freestanding hybrid electronic sheet according to a specific embodiment;

FIG. 12 is an image of a sample having only a single-walled carbon nanotube without a phage;

FIG. 13 is a scanning electron microscopic (SEM) image showing a nanostructure of a phage-bound hybrid electronic sheet according to an exemplary embodiment and a nanostructure of a non-phage bound electronic sheet;

FIGS. 14A and 14B are graphs showing electrochemical property of the hybrid electrode according to a specific embodiment;

FIG. 15 is a schematic illustration showing a fabrication process of a transparent flexible multi-layered hybrid electronic sheet-GOx-based biosensor according to a specific embodiment;

FIG. 16 is a CV graph showing a comparison of direct electron transfer (DET) reaction between a single-layered hybrid electronic sheet-GOx based biosensor according to an exemplary embodiment and a GOx electrode formed on a gold electrode without the hybrid electronic sheet;

FIG. 17 is a graph showing a comparison between a current response to glucose and a current response to a mixture of glucose with ascorbic acid and uric acid in the single-layered hybrid electronic sheet-GOx based biosensor according to a specific embodiment;

FIGS. 18A and 18B are graphs showing changes in pure DET current response according to a voltage scan rate in the single-layered hybrid electronic sheet-GOx based biosensor according to a specific embodiment;

FIG. 19A is a graph showing that DET redox of GOx increases with increasing concentration of immobilized GOx when different concentrations of GOx are immobilized on the hybrid electronic sheet according to a specific embodiment;

FIG. 19B is a graph showing that a current response to a change in glucose concentration increases with increasing concentration of GOx immobilized on the hybrid electronic sheet according to a specific embodiment, leading to an increase in sensor sensitivity;

FIG. 20A is a graph showing that a high DET reduction current increases in the multi-layered hybrid electronic sheet-GOx-based biosensor according to a specific embodiment, compared to the single-layered structure;

FIG. 20B is a graph showing that sensor sensitivity may be increased by multi-stacking, in which sensitivity to a change in glucose concentration increases in the multi-layered hybrid electronic sheet-GOx-based biosensor according to a specific embodiment, compared to the single-layered structure;

FIGS. 21A and 21B are graphs showing results of measuring sensitivity of the multi-layered hybrid electronic sheet-GOx-based biosensor according to an exemplary embodiment in a reference electrode harmless to human body;

FIGS. 22A and 22B are graphs showing sensitivity and flexibility of the transparent flexible multi-layered hybrid electronic sheet-GOx-based biosensor according to a specific embodiment;

FIG. 23A is a graph showing sensitivity of a hybrid electronic sheet-cholesterol oxidase-based biosensor according to a specific embodiment;

FIG. 23B is a graph showing sensitivity of a hybrid electronic sheet-lactate oxidase-based biosensor according to a specific embodiment;

FIG. 24A is a graph showing sensitivity of a hybrid electronic sheet-HRP-based biosensor according to a specific embodiment;

FIG. 24B is a graph showing sensitivity of a hybrid electronic sheet-catalase-based biosensor according to a specific embodiment;

FIG. 25A is a graph showing sensitivity of a hybrid electronic sheet-galactose oxidase-based biosensor according to a specific embodiment;

FIG. 25B is a graph showing sensitivity of a hybrid electronic sheet-tyrosinase-based biosensor according to a specific embodiment; and

FIG. 25C is a graph showing sensitivity of a hybrid electronic sheet-laccase-based biosensor according to a specific embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the exemplary embodiments are merely described below, by referring to the figures, to explain aspects of the present description.

An aspect provides a biosensor including a substrate; an electronic sheet formed on the substrate; and an analyte-binding material immobilized on the electronic sheet, in which the electronic sheet includes a graphitic material and a phage binding to the graphitic material, and the binding of the graphitic material and the phage occurs between a peptide displayed on a coat protein of the phage or a fragment thereof and the graphitic material.

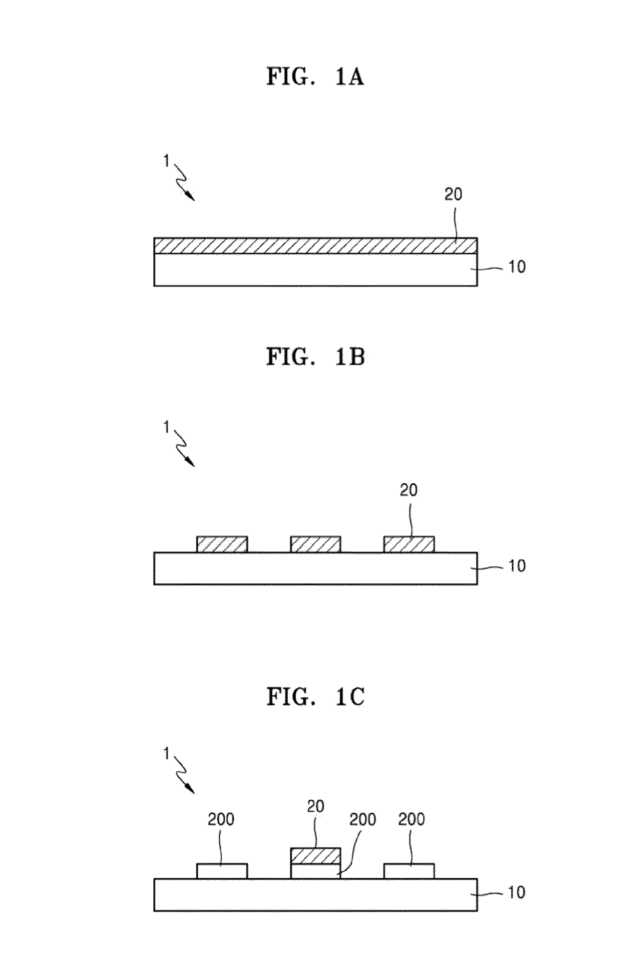

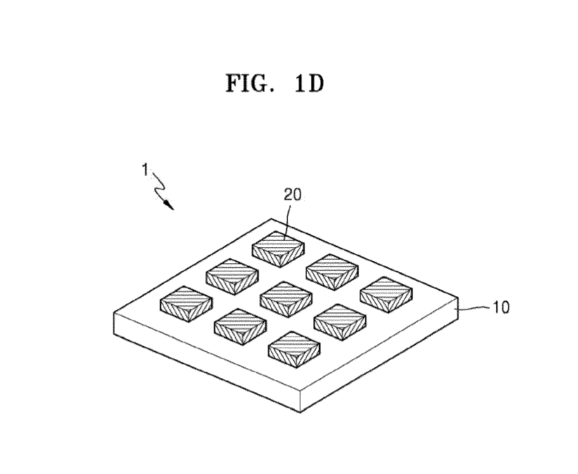

Referring to FIGS. 1A to 1D, a biosensor according to an embodiment includes a substrate 10 and an electronic sheet 20 located on the substrate. Referring to FIG. 1A, the electronic sheet 20 may be transferred on the substrate 10, and referring to FIGS. 1B and 1C, the electronic sheet 20 transferred on the substrate 10 may have a pattern. The electronic sheet 20 may be patterned without chemical etching. The substrate 10 may be a conductive substrate or an insulating substrate, and referring to FIG. 1D, the substrate 10 may be an insulating substrate with at least one electrode 200 disposed thereon. The substrate 10 may be a conductive substrate or an insulating substrate, or an insulating substrate with at least one electrode disposed thereon. The at least one electrode may include at least one electrode selected from a first electrode, a second electrode, or a third electrode. In some embodiments, the at least one electrode may include at least one electrode selected from a working electrode, an opposite electrode, and a reference electrode. In some embodiments, the at least one electrode may further include, the working electrode, the opposite electrode, and the reference electrode, at least one electrode selected from an auxiliary electrode and a recognition electrode. When an electronic sheet is formed on an insulating substrate with at least one electrode disposed thereon, the electronic sheet may be disposed on the first electrode, or the working electrode, or at least a portion thereof.

In some embodiments, the biosensor may further include a second substrate configured to face the substrate. The second substrate and the substrate may be identical to or different from each other. In a case in which the biosensor further includes the second substrate, the first electrode may face the second electrode.

Examples of the substrate may include a silver substrate, a silver epoxy substrate, a palladium substrate, a copper substrate, a gold substrate, a platinum substrate, a silver/silver chloride substrate, a silver/silver ion substrate, a mercury/mercury oxide substrate, a conductive carbon substrate, a semiconductor substrate, an oxide substrate, and a polymer substrate.

The substrate may be also a transparent flexible substrate. Examples of the transparent flexible substrate may include substrates that are manufactured from polydimethylsiloxane (PDMS), polyethersulfone (PES), poly(3,4-ethylenedioxythiophene), poly(styrenesulfonate), polyimide, polyurethane, polyester, perfluoropolyether (PFPE), polycarbonate, or combinations thereof.

The electronic sheet includes the graphitic material and the phage binding to the graphitic material, and the binding occurs between the peptide displayed on the coat protein of the phage or the fragment thereof and the graphitic material.

The term “sheet” used herein refers to a material having a certain width and a certain thickness, and may be understood as a concept including a film, a web, a film, or a composite structure thereof.

The electronic sheet may be formed having a pattern that is formed by using a substrate or a mask. One of ordinary skill in the art may pattern he electronic sheet according to purpose.

The electronic sheet may have an area of, for example, 0.0001 to 1000 cm², 0.0001 to 100 cm², or 1 to 20 cm², and a thickness of, for example, 20 to 400 nm, 40 to 200, or 40 to 100 nm. In addition, the internal structure of the graphitic material may have a percolated network structure. As used herein, the term “percolated network” may refer to a lattice structure consisting of random conductive or non-conductive linkages.

As used herein, the term “graphitic material” may refer to a material having a surface with hexagonal arrangement of carbon atoms, i.e., a graphitic surface, and may include any graphitic material having the graphitic surface, regardless of physical, chemical or structural properties. Examples thereof may include a graphene sheet, a highly ordered pyrolytic graphite (HOPG) sheet, a carbon nanotube such as a single-walled carbon nanotube, a double-walled carbon nanotube, and a multi-walled carbon nanotube, or fullerene. The graphitic material may be a metallic, semiconductive, or hybrid material. For example, the graphitic material may be a mixture of a graphene sheet and a single-walled carbon nanotube.

The peptide having a binding affinity specifically to the graphitic material may be a peptide or a peptide set including one or more selected from the group consisting of amino acid sequences of X₂SX₁AAX₂X₃P (SEQ ID NO. 1), X₂X₂PX₃X₂AX₃P (SEQ ID NO. 2), SX₁AAX₂X₃P (SEQ ID NO. 3) and X₂PX₃X₂AX₃P (SEQ ID NO. 4). In some embodiments, the peptide or peptide set may include one or more selected from the group consisting of amino acid sequences of SEQ ID NOS. 5 to 8. Consecutive amino acid sequences of a coat protein of a phage may be linked to the N-terminus or C-terminus of the amino acid sequence of the peptide or peptide set. Therefore, for example, the peptide or peptide set may have an amino acid sequence having a length of 5 to 60, 7 to 55, 7 to 40, 7 to 30, 7 to 20, or 7 to 10 amino acids.

The peptide may have a conservative substitution of a known peptide. As used herein, the term “conservative substitution” denotes replacement of a first amino acid residue by a second different amino acid residue without changing biophysical properties of a protein or a peptide. Here, the first and second amino acid residues mean those having side chains having similar biophysical properties. The similar biophysical properties may include an ability to donate or accept hydrophobicity, charge, polarity, or hydrogen bonding. Examples of the conservative substitution may be within the groups of basic amino acids (arginine, lysine, and histidine), acidic amino acids (glutamic acid and aspartic acid), polar amino acids (glutamine and asparagine), hydrophobic amino acids (leucine, isoleucine, valine and methionine), hydrophilic amino acids (aspartic acid, glutamic acid, asparagine and glutamine), aromatic amino acids (phenylalanine, tryptophan, tyrosine and histidine), and small amino acids (glycine, alanine, serine and threonine). Amino acid substitutions that do not generally alter specific activity are known in the art. For example, in the peptide, X1 may be W, Y, F or H, X2 may be D, E, N or Q, and X3 may be I, L or V.

The peptide may be selected from peptide libraries, for example, by a phage display technique. Through the phage display technique, the peptide may be genetically linked to, inserted into, or substituted for the coat protein of the phage, resulting in display of the protein on the exterior of phage, in which the peptide may be encoded by genetic information in the virion. Vast numbers of variants of the protein may be selected and screened by the displayed protein and DNA encoding the same, this method is called “biopanning”. Briefly, biopanning is carried out by incubating the pool of phage-displayed variants with a target (e.g., graphitic material) that has been immobilized, washing away unbound phage, and eluting specifically bound phage by disrupting the binding interactions between the phage and the target. A portion of the eluted phage is set aside for DNA sequencing and peptide identification, and the remainder is amplified in vivo to prepare a sub-library for the next round. Then, this procedure is repeated.

The term “phage” or “bacteriophage” is used interchangeably, and may refer to a virus that infects bacteria and replicates within the bacteria. The phage or bacteriophage may be used to display a peptide which selectively or specifically binds to a graphitic material or volatile organic compound. The phage may be genetically engineered to display the peptide capable of binding to the graphitic material on a coat protein of the phage or a fragment thereof. As used herein, the term “genetic engineering” or “genetically engineered” means introduction of one or more genetic modifications into the phage in order to display the peptide capable of binding to the graphitic material on the coat protein of the phage or the fragment thereof, or a phage prepared thereby. The genetic modifications include introduction of a foreign gene encoding the peptide. The phage may be a filamentous phage, for example, M13 phage, F1 phage, Fd phage, If1 phage, Ike phage, Zj/Z phage, Ff phage, Xf phage, Pf1 phage, or Pf3 phage.

As used herein, the term “phage display” or “phage with a peptide displayed thereon” may refer to a display of a functional foreign peptide or protein on the surface of a phage or phagemid particle. The surface of the phage may refer to a coat protein of the phage or a fragment thereof. The phage may be a phage in which the C-terminus of the functional foreign peptide is linked to the N-terminus of the coat protein of the phage, or the peptide is inserted between consecutive amino acid sequences of the coat protein of the phage or replaced for a part of the consecutive amino acid sequences of the coat protein. The positions in the amino acid sequence of the coat protein, at which the peptide is inserted or replaced, may be positions of 1 to 5, positions of 1 to 40, positions of 1 to 30, positions of 1 to 20, position of 1 to 10, positions of 2 to 8, positions of 2 to 4, positions of 2 to 3, positions of 3 to 4, or a position of 2 from the N-terminus of the coat protein.

In an exemplary embodiment, since the hybrid electronic sheet is bound with the phage displaying the peptide having a nondestructive binding ability, it has superior electrical property and also semiconductor property, and if necessary, the property is controllable.

In another specific embodiment, since the hybrid electronic sheet is structurally stable, transparent, and flexible, it may be transferred to various substrates or non-conventional substrates, and various patterns may be also formed thereon using a substrate or a mask.

In still another specific embodiment, since the hybrid electronic sheet is hybridized with the phage, it is highly compatible with biomaterials, and it may be further functionalized with other biomaterials.

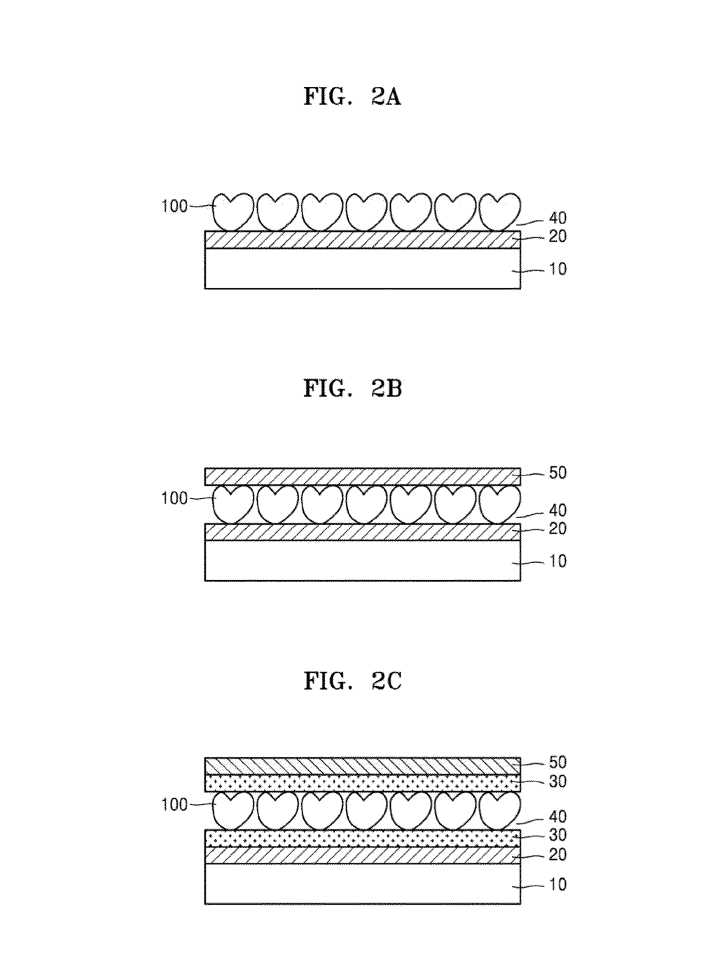

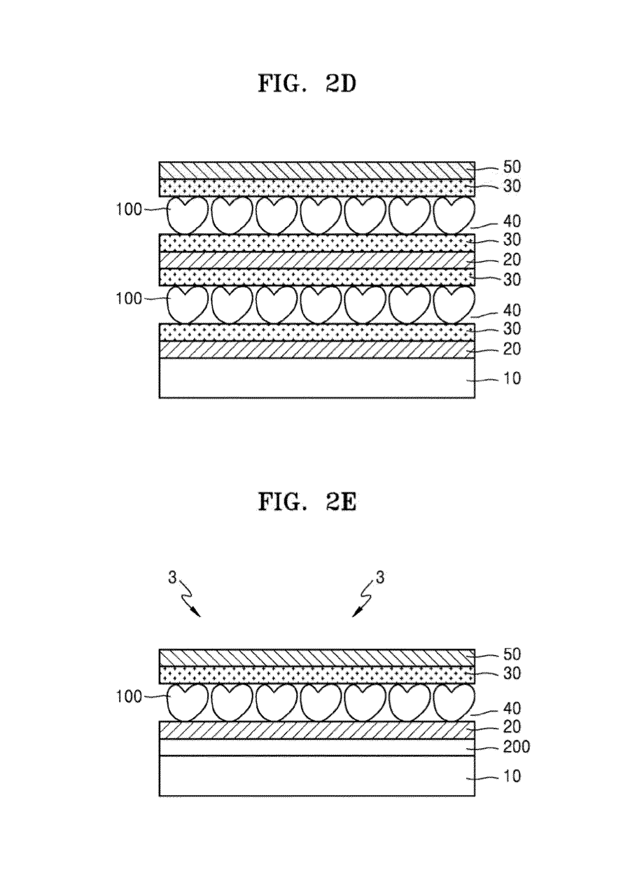

Further, referring to FIG. 2A, the biosensor according to an exemplary embodiment includes an analyte-binding material 100 which is immobilized on the electronic sheet.

As used herein, the term “analyte-binding material” or “analyte-binding reagent” may be used interchangeably, and may refer to a material capable of providing the electronic sheet with functionalization or a material capable of specifically binding to an analyte. The analyte-binding material may include a redox enzyme. The redox enzyme may refer to an enzyme oxidizing or reducing a substrate, and example thereof may include oxidase, peroxidase, reductase, catalase, and dehydrogenase. Example of the redox enzyme may include glucose oxidase, lactate oxidase, cholesterol oxidase, glutamate oxidase, horseradish peroxidase (HRP), alcohol oxidase, glucose oxidase (GOx), glucose dehydrogenase (GDH), cholesterol esterase, ascorbic acid oxidase, alcohol dehydrogenase, laccase, tyrosinase, galactose oxidase, and bilirubin oxidase. The analyte-binding material may be immobilized on the electronic sheet, and the term “immobilized” may refer to a chemical or physical binding between the analyte-binding material and the electronic sheet.

As used herein, the term “analyte” may refer to a material of interest which may be present in a sample. The detectable analyte may include materials involved in a specific binding interaction with one or more analyte-binding materials, which participate in a sandwich, competitive, or replacement assay configuration. Examples of the analyte may include antigens such as peptides (e.g., hormone) or haptens, proteins (e.g., enzyme), carbohydrates, proteins, drugs, agricultural chemicals, microorganisms, antibodies, and nucleic acids participating in sequence-specific hybridization with complementary sequences. More specific examples of the analyte may include glucose, cholesterol, lactate, hydrogen peroxide, catechol, tyrosine, and galactose.

Referring to FIG. 2B, the biosensor may further include a protection layer 50 formed on an analyte-binding material 100 that is immobilized. The protection layer 50 may be any suitable layer that is used to protect a biosensor and that is known to one of ordinary skill in the art or is obvious in view of general knowledge in the art. For example, the protection layer 50 may be formed of a tetrafluoroethylene-based copolymer or Nafion®, or may be a second electronic sheet.

In some embodiments, the electronic sheet 20 or the protection layer 50 may be reformed such that a surface thereof contacting an analyte-binding material has a positive or negative charge that is opposite to that of the analyte-binding material.

Referring to FIG. 2C, the surface of the electronic sheet 20 or protection layer 50 is reformed to have a positive or negative charge by using a polymer 30 that has a positive or negative charge. When the analyte-binding material 100 has a negative charge, the surface of the electronic sheet 20 or protection layer 50 contacting the analyte-binding material 100 may be reformed to have a positive charge by using a positive-charge polymer, and when the analyte-binding material 100 has a positive charge, the surface of the electronic sheet 20 or protection layer 50 may be reformed by using a negative-charge polymer 30. When the analyte-binding material 100 has a positive charge, the surface of the electronic sheet 20 or protection layer 50 is reformed by sequentially using a positive-charge polymer and a negative-charge polymer in this stated order. By doing so, the analyte-binding material 100 having a positive charge may be immobilized on or bound to the electronic sheet 20.

Examples of the positively charged polymer may include PAH (Poly(allyamine)), PDDA (Polydiallyldimethylammonium)), PEI (Poly(ethyleneimine)), and PAMPDDA (Poly(acrylamide-co-diallyldimethylammonium). Further, examples of the negatively charged polymer may include PSS (Poly (4-styrenesulfonate), PAA (Poly(acrylic acid)), PAM (Poly(acryl amide)), Poly(vinylphosphonic acid), PAAMP (Poly(2-acrylamido-2-methyl-1-propanesulfonic acid), PATS (Poly(anetholesulfonic acid)), and PVS (Poly(vinyl sulfate)).

In some embodiments, referring to FIG. 2D, the biosensor may include a plurality of repeating units, each repeating unit including the electronic sheet 20 and either the analyte-binding material 100 or the analyte-binding layer 40. The number of repeating units used herein may be 2 or more, 3 or more, 4 or more, 5 or more, 6 or more, 7 or more, or 8 or more. In some embodiments, a biosensor including a plurality of the repeating units may have higher sensibility than a biosensor including a single unit.

Referring to FIG. 2E, an electronic sheet with an analyte-binding material immobilized thereon is disposed on an insulating substrate with at least one electrode disposed thereon as illustrated in FIG. 1D. The at least one electrode may include at least one electrode selected from a first electrode, a second electrode, or a third electrode. In some embodiments, the at least one electrode may include at least one electrode selected from a working electrode, an opposite electrode, and a reference electrode. In some embodiments, the at least one electrode may further include, the working electrode, the opposite electrode, and the reference electrode, at least one electrode selected from an auxiliary electrode and a recognition electrode. When an electronic sheet is formed on an insulating substrate with at least one electrode disposed thereon, the electronic sheet may be disposed on the first electrode, or the working electrode, or at least a portion thereof.

The biosensor may further include a test cell for accommodating a sample, an electronic sheet, and an analyte-binding material, and the test cell may include a channel having an inlet for accepting a sample or an outlet for discharging the sample.

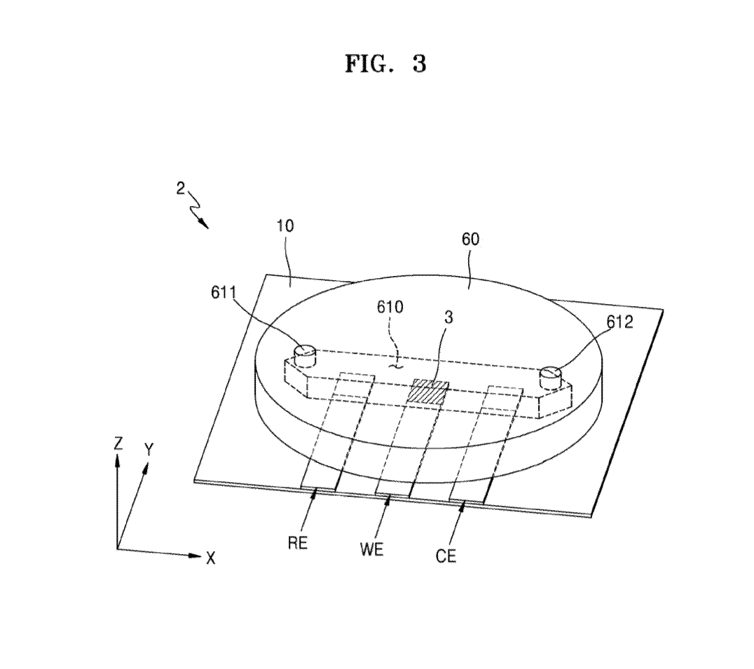

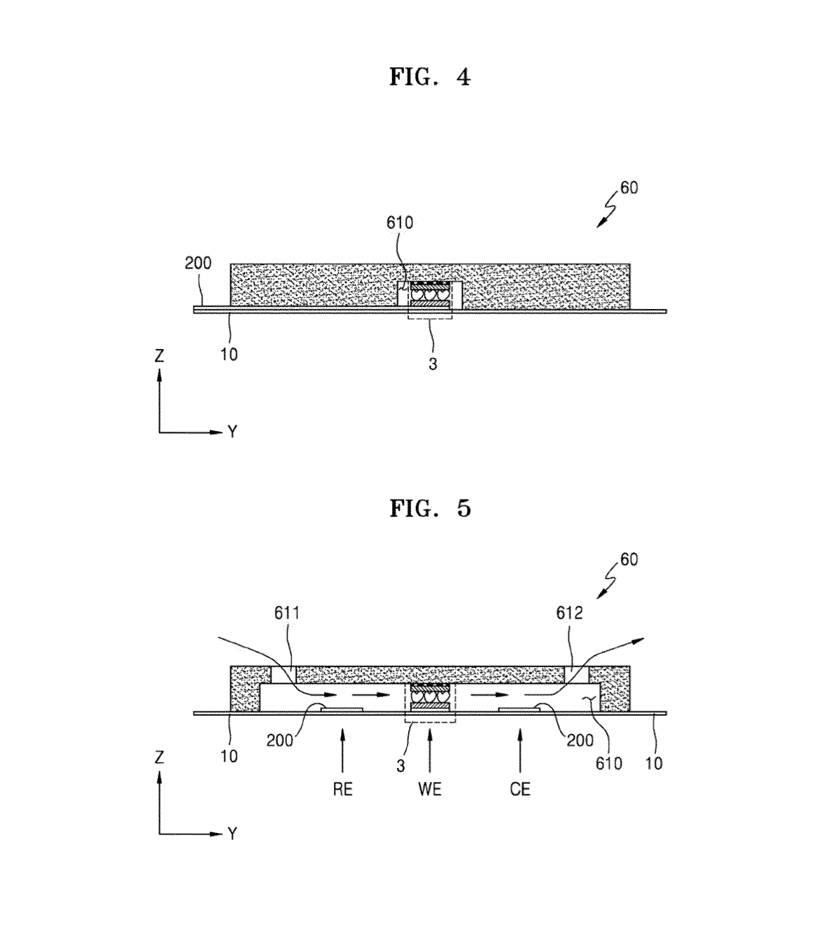

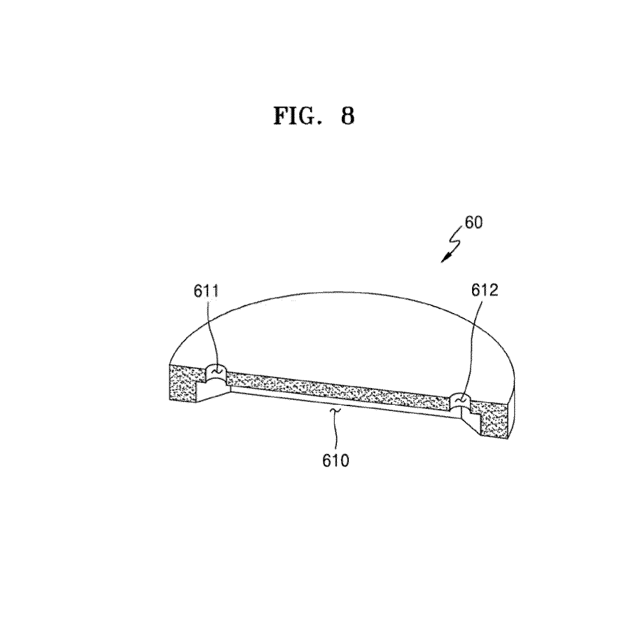

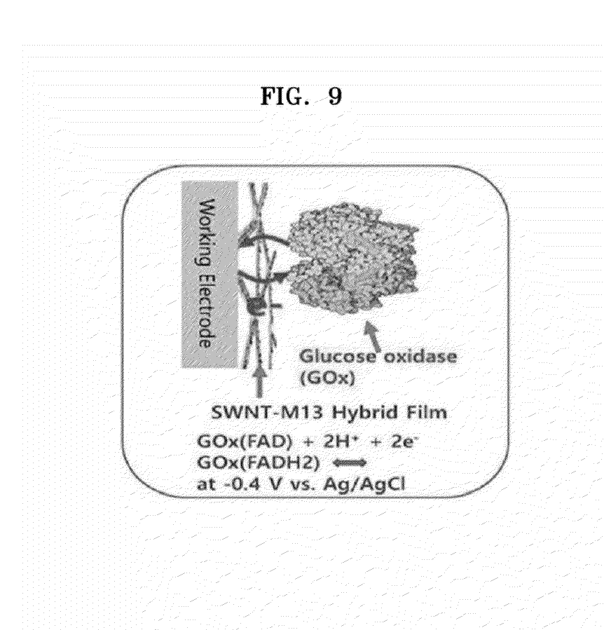

Referring to FIGS. 3 to 8, a biosensor 2 according to an embodiment may include a substrate 10 with a working electrode WE, an opposite electrode CE, and a reference electrode RE disposed thereon, and a test cell 610 having a channel. The test cell 610 may be covered by a cover 60. The test cell 610 may include an inlet 611 for accepting a sample or an outlet 612 for discharging the sample. The sample may enter through the inlet 611, and an analyte included in the sample may experience an redox-reaction together with an analyte-binding material to cause an electrochemical gradient in the test cell 610. The “chemical potential gradient” may mean a concentration gradient of a redox-active material. When the gradient is present between two electrodes, a potential difference may be detectable when a circuit is opened, and when the circuit is closed, a current may flow until the gradient is reduced to zero. The chemical potential gradient may be a redox enzyme, for example, a potential difference between electrodes stemming from an asymmetry of the analyte-binding material distribution or a potential gradient occurring due to the providing of a current flow. In a biosensor according to an embodiment, in the case of a working electrode with the electronic sheet 20 transferred thereon, a strong peak of the redox reaction may occur, and otherwise, the redox peak slightly occurs or does not occur. Accordingly, as illustrated in FIG. 9, in a biosensor according to an embodiment, the migration of electrons due to the redox reaction between an analyte and the analyte-binding material 100 may be a direct electron transfer (DET) on a working electrode with an electronic sheet transferred thereon in the absence of a medium.

The channel in the test cell may be modified to facilitate capillary action of a sample. The modification may be performed using hydrophobic materials. Examples of the hydrophobic materials may include glyceride, polystyrene, polymethyl methacrylate (PMMA), polyethylene terephthalate (PET), polyvinyl chloride (PVC), polyethylene (PE), polypropylene (PP), polytetrafluoroethylene (PTFE), silicic compound, wax, wax emulsion, aliphatic polyester-based polymers such as poly(L-lactic acid) (PLLA), poly(D,L-lactic acid) (PDLLA), poly(glycolic acid) (PGA), poly(carprolactone) (PCL), poly(hydroxyalkanoate), polydioxanone (PDS), or polytrimethylene carbonate, and copolymers thereof such as poly(lactic acid-co-glycolic acid) (PLGA), poly(L-lactic acid-co-carprolactone) (PLCL), or poly(glycolic acid-co-carprolactone) (PGCL).

The biosensor according to an exemplary embodiment may further include a meter for determination of an analyte. As used herein, the term “determination of an analyte” refers to qualitative, semi-quantitative and quantitative processes for evaluating a sample. In a qualitative evaluation, a result indicates whether or not the analyte is detected in the sample. In a semi-quantitative evaluation, the result indicates whether or not the analyte is present above some predefined threshold. In a quantitative evaluation, the result is a numerical indication of the amount of the analyte present.

The meter may include an electronic device that measures a potential difference or current at a predetermined time point after a sample is introduced, and converts a measurement value into a numerical indication. The measuring of the potential difference or current may be determining of an oxidation current reaction voltage value by using cyclic voltammetry (CV). According to the CV, a potential of the first electrode (for example, a working electrode) is circulated at a predetermined rate to measure a current.

The converting of the measurement value may be performed by referring to a look-up table that is used to convert a specific value of a current or potential into a value of an analyte dependent on a specific device structure and a correction value with respect to the analyte. In some embodiments, the meter may further include a display showing results and a frame including at least one controlling interface (for example, a power button or a scroll wheel). The frame may include a slot for receiving a biosensor. The frame may include a circuit thereinside to apply a potential or current to an electrode included in the biocensor when a sample is provided. A suitable circuit for the meter may be a suitable voltage meter that measures a potential crossing the electrode. Also, provided is a switch that is opened when the potential is measured or is closed when the current is measured. The switch may be a mechanical switch (for example, relay), or a field-effect transistor (FET) switch, or a solid-state switch. The circuit may be used to measure a potential difference or a current difference. As understandable to one of ordinary skill in the art, other circuits including more simple or complicated circuits may be used to apply at least one selected from a potential difference and a current.

Another aspect provides a wearable device including the biosensor which includes the substrate; the electronic sheet formed on the substrate; and the analyte-binding material immobilized on the electronic sheet, in which the electronic sheet includes the graphitic material and the phage binding to the graphitic material, and the binding of the graphitic material and the phage occurs between the peptide displayed on a coat protein of the phage or the fragment thereof and the graphitic material.

The biosensor is the same as described above.

The wearable device may be used for detecting bioinformation. The wearable device may be a patch, a watch, or a contact lens.

The term “contact lens” may refer to any ophthalmic device or any device for cosmetic appearance, which resides in or on the eye. For example, contact lens may include an intraocular lens, an overlay lens, an ocular insert, a punctual plug, and other similar ophthalmic device through which vision is corrected or modified, an eye condition may be enhanced or prevented, and/or through which eye physiology is cosmetically enhanced (e.g., iris color). The contact lens may include soft contact lenses made from silicone elastomers or hydrogels (e.g., silicone hydrogels), and fluorohydrogels.

For logic communication with the biosensor and output of data related to control of the biosensor, the contact lens of detecting bioinformation according to an exemplary embodiment may further include a controller that receives and processes signal data generated from the biosensor.

The biosensor in the contact lens of detecting bioinformation is controlled by the controller, which responds at a predetermined time interval or to a particular event (e.g., remarkable decrease or increase of glucose in tear) to receive and process bioinformation detected by the biosensor.

Further, the contact lens of detecting bioinformation may further include a memory which stores a processor for controller movement and temporarily stores input/output data (e.g., bioinformation). The memory may store information about an analyte (e.g., glucose) in the tear, which is detected by the biosensor.

Further, the contact lens of detecting bioinformation may further include a wireless communication unit to transfer information processed by the controller or stored in the memory to a person wearing the contact lens or another user (e.g., doctor, hospital, wearer's family, etc.) who has a wireless communication system. For example, the wireless communication unit may include a broadcast reception module, a mobile communication module, a wireless internet module, and a near field communications module. Information about the analyte in the tear, which is detected by the biosensor, may be transferred to the wearer or another user via the wireless communication unit.

The contact lens of detecting bioinformation may further include an energy supply source capable of supplying energy or making the device under operation. The energy supply source may be, for example, a lithium ion battery.

Further, the biosensor, the controller, the memory, the wireless communication unit, or the energy supply source may be embedded in the contact lens or attached on the surface of the contact lens via a media insert.

In the wearable device according to a specific embodiment, the biosensor exhibits remarkable electrochemical property on a transparent flexible substrate and an electrode harmless to the human body. Further, the biosensor does not need a mediator harmful to the human body, and its sensitivity is high enough to detect a small amount of analyte in a sample. Thus, the biosensor may be usefully applied to a wearable device (e.g., contact lens of detecting bioinformation).

Hereinafter, the present invention will be described in more detail with reference to Examples. However, these Examples are for illustrative purposes only, and the invention is not intended to be limited by these Examples.

### Example 1

### Fabrication and Characterization of Biosensor

1. Preparation of Electrode Having Hybrid Electronic Sheet

1.1. Preparation of Colloid Solution

First, an aqueous solution is prepared by adding 2% w/v sodium cholate as a surfactant to distilled water, and a colloid solution is prepared by stabilizing single-walled carbon nanotube with the sodium cholate by dialysis of carbon nanotube (manufacturer: Nanointegris, SuperPure SWNTs, solution-type, concentration: 250 mg/ml) for 48 hours.

In this regard, assuming that an average length and an average diameter of the carbon nanotube (CNT) are 1 μm and 1.4 nm, respectively, the number of the single-walled carbon nanotube included in the colloid solution may be calculated according to the following equation.

Number of single-walled carbon nanotube (number/mL)=concentration (μg/mL)×3×10¹¹ CNT [Equation 1]

According to this Equation, the number of the single-walled carbon nanotube included in the colloid solution is calculated as 7.5×10¹³/mL.

### 1.2. Preparation of Phage Displaying Peptide Having Binding Ability to Graphitic Material

As M13 phages having a strong binding affinity to the graphitic surface, a phage (p8 GB#1) displaying a peptide DSWAADIP (SEQ ID NO. 5) having a strong binding affinity to the graphitic surface, a phage (p8 GB#5) displaying DNPIQAVP (SEQ ID NO. 6), and a phage displaying SWAADIP (SEQ ID NO. 7), and NPIQAVP (SEQ ID NO. 8) are prepared by the following method.

First, an M13HK vector is prepared using oligonucleotides of SEQ ID NOS. 10 and 11 for site-directed mutation of the 1381st base pair C of an M13KE vector (NEB, product#N0316S) (SEQ ID NO. 9) to G. The prepared M13HK vector is double-digested using restriction enzymes, BspHI (NEB, product# R0517S) and BamHI (NEB, product#R3136T), and dephosphorylated using antarctic phosphatase. The dephosphorylated vector is ligated to a double-digested DNA duplex by incubation at 16° C. overnight. A product is then purified and concentrated. Electorcompetent cells (XL-1 Blue, Stratagene) are transformed with 2 μl of a concentrated ligated vector solution by electroporation at 18 kV/cml. A total of five transformations are performed for the library construction. Then, the transformed cells are incubated for 60 minutes, and fractions of several transformants are plated onto agar plates containing x-gal/isopropyl-β-D-1-thiogalactopyranoside (IPTG)/tetracycline (Tet) to determine the diversity of the library. The remaining cells are amplified in a shaking incubator for 8 hours. Oligonucleotides of SEQ ID NOS. 12 and 13 are used in construction of the phage-display p8 peptide library.

The base sequences of the phage-display p8 peptide library constructed according to an exemplary embodiment have diversity of 4.8×10⁷ pfu (plaque forming unit), and include approximately 1.3×10⁵ copies of each sequence.

Then, a highly ordered pyrolytic graphite (HOPG) substrate (manufacturer: SPI product#439HP-AB) having a diameter of 1 cm was prepared. In this regard, the HOPG substrate is a HOPG substrate having a relatively large grain size of 100 μm or smaller. Previously, a carbon nanotube film surface damaged during its production process has been generally used as a graphitic surface, and thus it is difficult to identify peptides having high binding affinity. In order to solve this problem, a fresh surface is detached from HOPG as a material having a graphitic surface using a tape immediately before use, so as to minimize the defect of the sample surface due to, for example, oxidation. Subsequently, the phage display p8 peptide library of 4.8×10¹⁰ pfu (4.8×10⁷ diversities, 1000 copies per each sequence) prepared in 1 of Example 1 is prepared in 100 μL of Tris-buffered saline (TBS) and conjugated with the HOPG surface for 1 hour in a shaking incubator at 100 rpm. 1 hour later, the solution is removed and the surface is washed 10 times in TBS. The washed HOPG surface is reacted with Tris-HCl of pH 2.2 as an acidic buffer for 8 minutes to elute peptides reacting non-selectively, and the remaining phage was eluted with an XL-1 blue E. coli culture in mid-log phase for 30 minutes. A portion of the eluted culture is set aside for DNA sequencing and peptide identification, and the remainder is amplified to prepare a sub-library for the next round. The above procedure is repeated using the prepared sub-library. Meanwhile, the left plaque is subjected to DNA sequencing to obtain the p8 peptide sequence, and the sequence is analyzed to obtain a phage (P8 GB#1) with DSWAADIP (SEQ ID NO: 5) displayed thereon, a phage (p8 GB#5) with DNPIQAVP (SEQ ID NO: 6) displayed thereon, a phage with SWAADIP (SEQ ID NO: 7) displayed thereon, and a phage with NPIQAVP (SEQ ID NO: 8) displayed thereon. Herein, DSWAADIP (SEQ ID NO: 5), DNPIQAVP (SEQ ID NO: 6), SWAADIP (SEQ ID NO: 7), and NPIQAVP (SEQ ID NO: 8) are peptide sequences having a strong binding affinity to a graphitic material.

### 1.3 Preparation of Electrode Having Hybrid Electronic Sheet

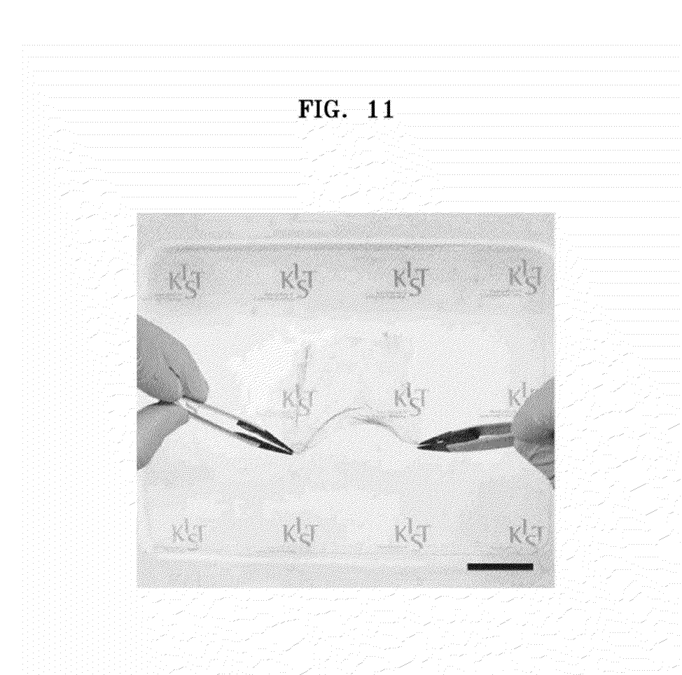

The prepared colloid solution and the phage solution containing M13 phage (p8 GB#1) having a strong binding affinity to the graphitic surface are mixed at a molar ratio of 4:1. Next, for dialysis, the mixture is added to a semipermeable dialysis membrane (SpectrumLab, MWCO 12,000-14,000, product #132 700) tube, and the membrane tube is dialyzed against triple distilled water. About 16 hours after the dialysis, a thin electronic sheet is formed along the surface of the membrane tube. Next, the membrane tube is transferred to triple distilled water and the electronic sheet is detached by twisting the membrane of the membrane tube and then dried. The prepared electronic sheet has a thickness of about 100 nm. FIG. 11 is an image of the electronic sheet formed by mixing at a molar ratio of 4:1. Thereafter, the prepared freestanding hybrid electronic sheet film is placed on a commercial gold electrode (SPE 250BT, DropSens) having a diameter of 4 mm using a stencil mask having a desired 4 mm-diameter pattern, and then dried in air for 1 hour. After removing the stencil mask, the hybrid film transferred onto the gold electrode is washed with deionized water, and then dried using nitrogen gas. The hybrid film thus prepared has a thickness of about 200 nm.

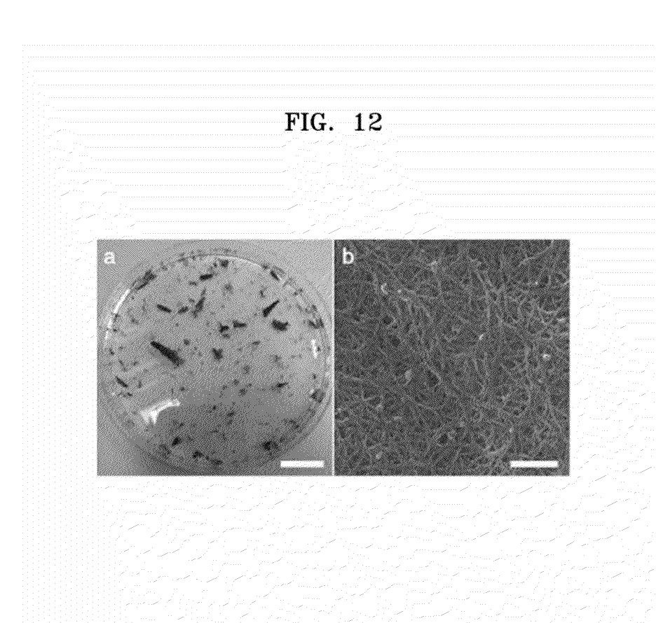

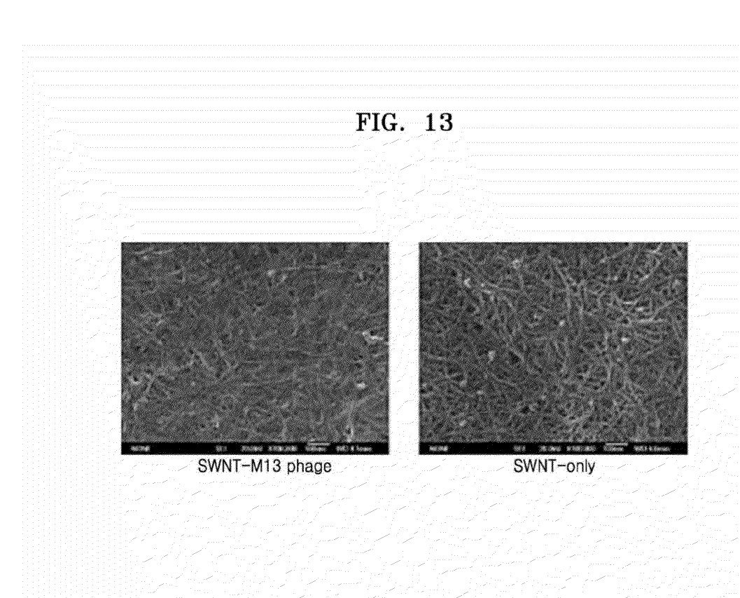

As Comparative Example, an electronic sheet including no phage is prepared as follows. First, an aqueous solution is prepared by adding 2% w/v sodium cholate as a surfactant to distilled water, and a colloid solution is prepared by stabilizing a single-walled carbon nanotube with the sodium cholate by dialysis of the single-walled carbon nanotube (manufacturer: Nanointegris, SuperPure SWNTs, solution-type, concentration: 250 μg/ml) as the graphitic material for 48 hours. Next, for dialysis, 0.4 mL of the colloid solution diluted with 10 mL of 1% w/v sodium cholate aqueous solution is added to a semipermeable dialysis membrane (SpectrumLab, MWCO 12,000-14,000, product #132 700) tube, and the membrane tube is dialyzed against triple distilled water. About 24 hours after the dialysis, an electronic sheet is formed along the surface of the membrane tube. Next, the membrane tube is transferred to triple distilled water and the electronic sheet is detached by twisting the membrane of the membrane tube. FIG. 12 shows a photograph and a scanning electron microscopic (SEM) image of the detached electronic sheet, compared with the phage-bound hybrid electronic sheet of FIG. 11. Further, SEM images of nanostructures of the phage-bound hybrid electronic sheet and the non-phage-bound electronic sheet are compared and the result is shown in FIG. 13.

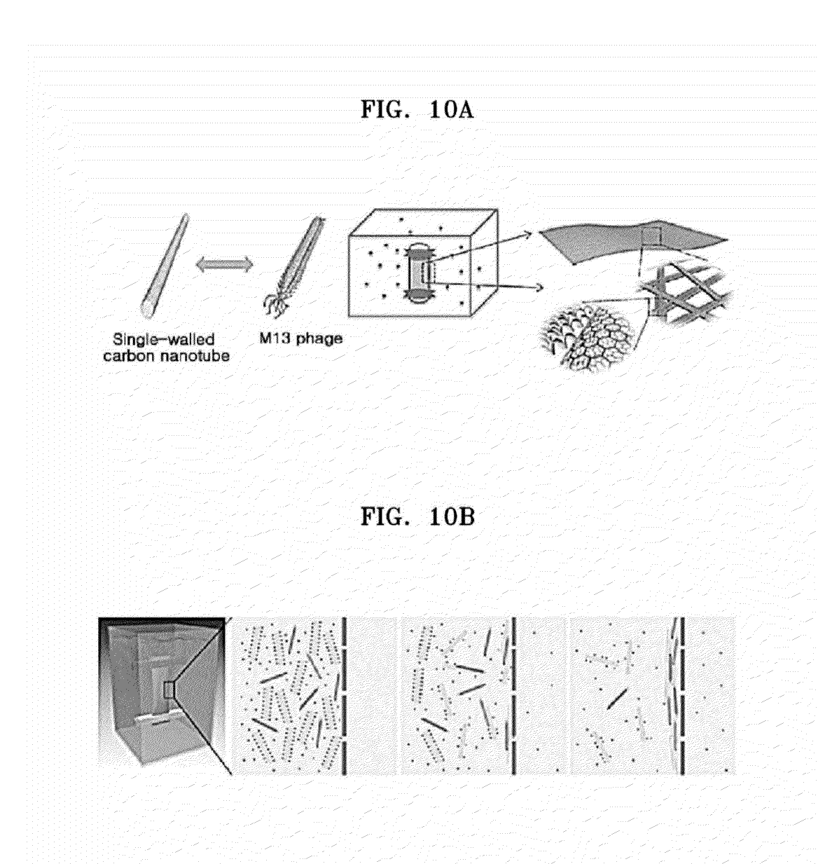

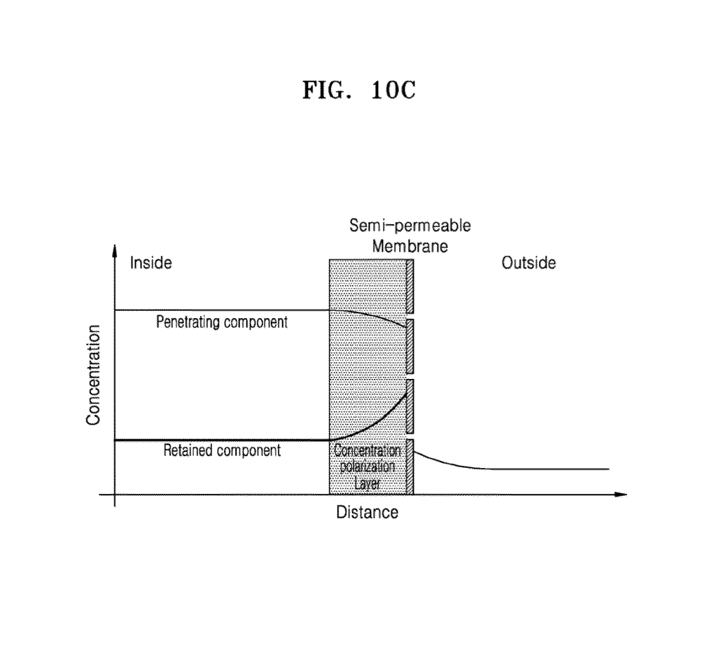

FIG. 10A is a schematic illustration of a production process of the hybrid electronic sheet according to an exemplary embodiment.

As shown in FIG. 10A, carbon nanotube is dispersed or dissolved in the colloid material which is stabilized by adding it to the surfactant-containing solution. Single-walled carbon nanotube is bound with about one M13 phage finally to form a sheet having a percolated network structure of carbon nanotube and M13 phage.

FIG. 10B is a schematic illustration of a formation principle of the hybrid electronic sheet according to an exemplary embodiment.

FIG. 10C is a graph showing concentration polarization in the formation principle of the hybrid electronic sheet according to an exemplary embodiment.

Referring to FIGS. 10B and 10C, formation of the carbon nanotube bound with M13 phage displaying the peptide according to an exemplary embodiment may be achieved by adding the mixture of the phage solution and the colloid solution to the membrane tube, followed by dialysis against the dialysis solution. While the dialysis proceeds, the concentration of the surfactant, which is attached on the surface of the carbon nanotube in the colloid material and stabilizes the carbonaceous material, in the tube decreases due to diffusion owing to a concentration difference inside and outside the membrane. This diffusion-driven dilution is the most prominent near the membrane. Since the M13 phage displaying the peptide having strong binding affinity to carbon nanotube can begin reacting with the carbon nanotube only when the concentration of the surfactant surrounding the carbon nanotube is low, the binding occurs near the membrane where the dilution occurs the most actively, when the dialysis proceeds for a predetermined time. Based on this principle, a sheet may be formed through dialysis.

FIG. 11 is an image of a large-area freestanding hybrid electronic sheet according to a specific embodiment.

FIG. 12 is an image of a sample having only a single-walled carbon nanotube without a phage.

FIG. 13 is a scanning electron microscopic (SEM) image showing a nanostructure of a phage-bound hybrid electronic sheet according to an exemplary embodiment and a nanostructure of a non-phage bound electronic sheet.

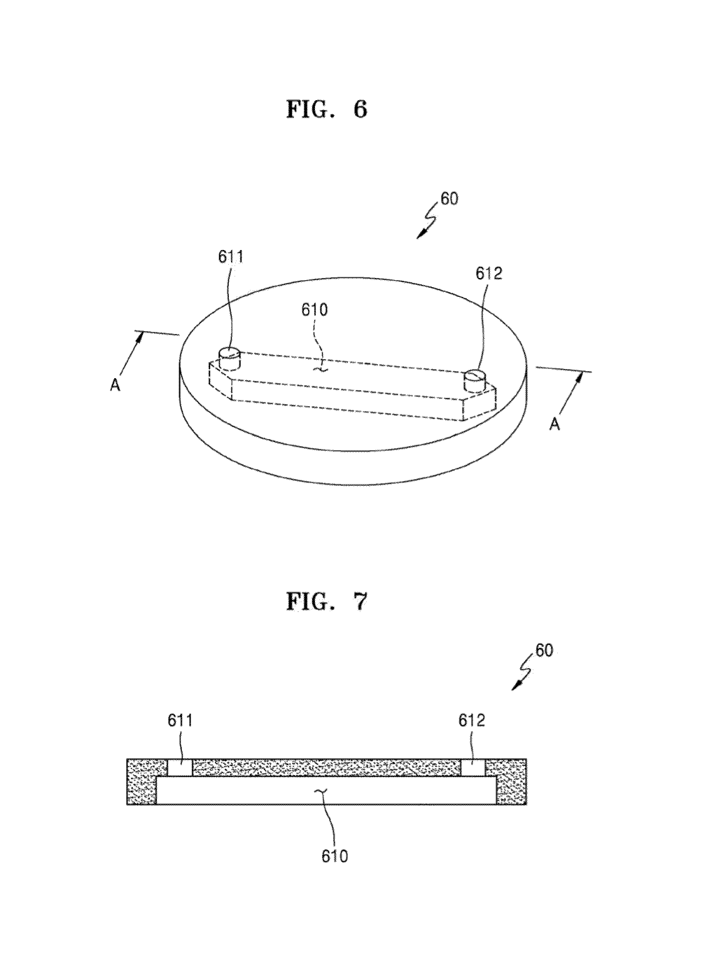

As shown in FIGS. 11 through 13, the phage-bound hybrid electronic sheet according to an exemplary embodiment is stably formed with a large area due to binding of the carbon nanotube and the phage and has a nanostructure in which the carbon nanotubes are uniformly distributed. In contrast, as shown in FIG. 6, non-phage-bound electronic sheet is broken into pieces during the preparation process and has a microstructure with bundling. These results indicate that the freestanding phage-bound hybrid electronic sheet according to an exemplary embodiment maintains its shape owing to the strong binding affinity between the carbon nanotube and the phage, whereas the electronic sheet is formed along the membrane but broken easily when dialysis is performed without addition of the phage, which is a limitation in its application.

1.4. Test of Electrochemical Property of Electrode Having Hybrid Electronic Sheet

To test electrochemical property of the electrode prepared in 1.1.3 of Example 1, a gold collector electrode (SPE 250BT, DropSens) is purchased and used as Comparative Example without surface modification.

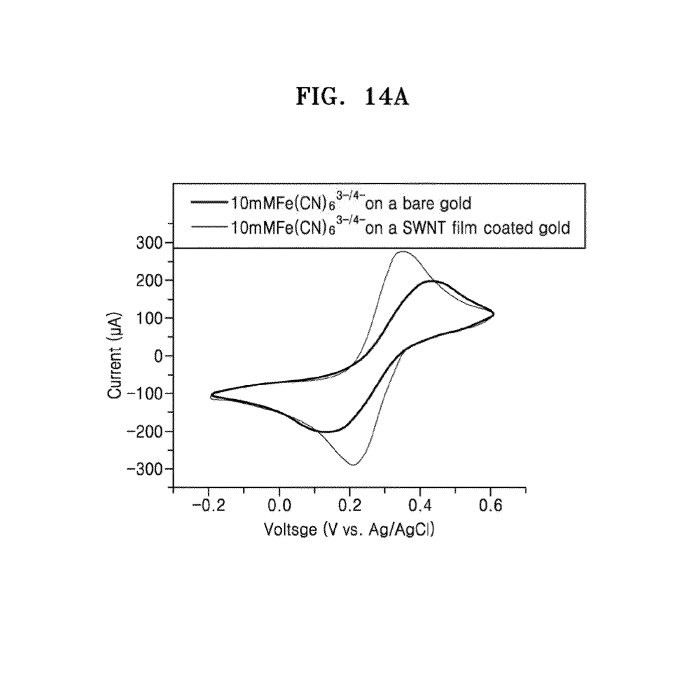

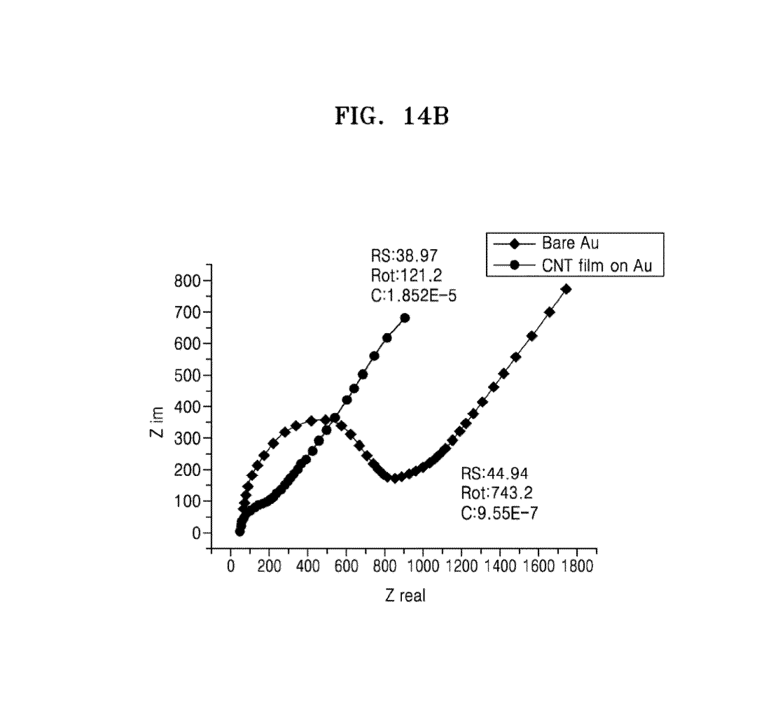

In detail, the electrode prepared in 1.1.3 of Example 1 and the electrode of Comparative Example are used as working electrodes (WE), a Pt-coated titanium chamber is used as a counter electrode (CE), and Ag/AgCl (3M KCl saturated, PAR, K0260) is used as a reference electrode (RE), and 10 mM K₃[Fe(CN)₆] (244023, Sigma Aldrich) is mixed with 10 mM PBS buffer (pH=7.4, 79383, Sigma Aldrich) as an electrolyte to measure redox reactions by cyclic voltammetry (CV). The measurement voltage is applied in a range of −0.2 V˜0.6 V versus the reference electrode at a scan rate of 200 mV/s. The result is shown in FIG. 14a. Further, 10 mV AC is applied to the electrodes in a range of 100,000 Hz˜0.1 Hz and electrochemical impedance spectroscopy is performed at the open circuit potential (OCP) of an electrochemical system cell. The result is shown in FIG. 14B.

FIGS. 14A through 14B are graphs showing electrochemical property of the hybrid electrode according to a specific embodiment.

As shown in FIG. 14A, the hybrid electronic sheet-transferred electrode shows about 50% increase in a redox current, compared to the gold electrode of Comparative Example.

As shown in FIG. 14B, the hybrid electronic sheet-transferred electrode shows about 80% decrease in charge transfer resistance (Rct), compared to the gold electrode of Comparative Example.

These results indicate that electrochemical activity of the hybrid electrode according to an exemplary embodiment is higher than that of the gold electrode which is commonly used in electrochemical experiments. These results show that the hybrid electronic sheet has a thickness of about 200 nm and also suggest that the hybrid electrode according to an exemplary embodiment can be used as a high-performance biosensor.

2. Fabrication and Characterization of Hybrid Electronic Sheet-GOx-Based Biosensor

2.1. Fabrication of Single-Layered Hybrid Electronic Sheet-GOx-Based Biosensor

2.1.1. Fabrication and Characterization of Single-Layered Hybrid Electronic Sheet-GOx-Based Biosensor 1

10 μl of a solution containing 6 mg of positively charged poly-allyamine hydrochloride (PAH, 43092, MW: 120000-200000, Alfa Aesar) is added dropwise onto the negatively charged hybrid electrode prepared in 1.3 of Example 1, and then dried in air for 1 hour. After drying, the electrode is washed with deionized water and dried using nitrogen gas. 20 μl of a solution prepared by dissolving 30 mg of GOx (Glucose oxidase) in 1 mL of PBS buffer solution is added dropwise thereto, and allowed for immobilization in a refrigerator at 4° C. for 12 hours. After immobilization, the electrode is carefully washed with 10 mM PBS buffer, and then 10 μl PAH is added dropwise thereto. The electrode is dried in air for 1 hour. After drying, the electrode is washed with deionized water and dried using nitrogen gas. To protect the immobilized GOx layer, the layer is further coated with 1 μl of 5% Nafion (70160, Sigma Aldrich) or another hybrid electronic sheet as a protection layer.

2.1.2. Fabrication and Characterization of Single-Layered Hybrid Electronic Sheet-GOx-Based Biosensors 2 to 5

To analyze sensitivity according to the concentration of the immobilized GOx, 4 single-layered hybrid electronic sheet-GOx-based biosensors are further fabricated in the same manner as in 2.1.1, except for using GOx at different concentrations of 10 mg/mL, 25 mg/mL, 50 mg/mL and 100 mg/mL.

2.1.3. Fabrication of Comparative Example

A GOx-immobilized biosensor is fabricated in the same manner as in 2.1.1, except that the hybrid electronic sheet prepared in 1.3 is not transferred. This biosensor is used as Comparative Example of the biosensor according to a specific embodiment.

2.2. Fabrication of Multi-Layered Hybrid Electronic Sheet-GOx-Based Biosensor

The procedure of Example 2.1.1 is further repeated to form a Gold-(hybrid film/PAH/GOx/PAH)₂ structure. 1 μl of 5% Nafion (70160, Sigma Aldrich) or another hybrid electronic sheet as a protection layer is further applied to the top PAH layer so as to fabricate a multi-layered hybrid electronic sheet-GOx-based biosensor.

2.3. Fabrication of Transparent Flexible Multi-Layered Hybrid Electronic Sheet-GOx-Based Biosensor

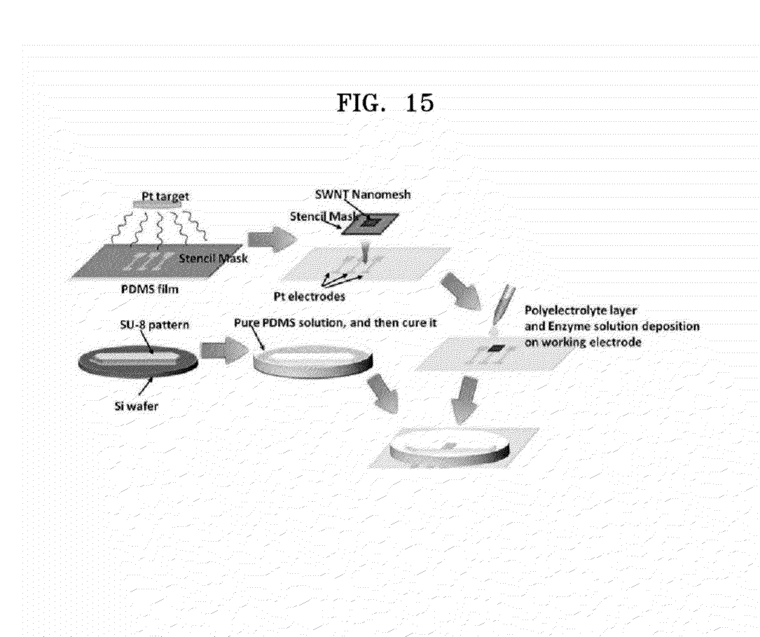

To fabricate a transparent flexible multi-layered hybrid electronic sheet-GOx-based biosensor, a process is performed as illustrated in FIG. 15.

In detail, a platinum electrode having a thickness of 100 nm and a size of 2 mm×2 mm is deposited by sputtering on a polydimethylsiloxane (PDMS) film having a size of 5 cm×2.5 cm which is covered with a stencil mask. The middle electrode of three platinum electrodes is used as a working electrode (WE), and the left and right electrodes are used as a counter electrode (CE) and a pseudo-reference electrode (RE), respectively. Further, the electronic sheet of Example 1.1.3 is connected to the working electrode using a stencil mask. Thereafter, positively charged PEI (polyethylene imine) is applied onto the electronic sheet, and then 2 μl of 100 mg/ml GOx is immobilized thereon. This process is repeated once, so that the platinum working electrode on PDMS is fabricated to have a structure of (GOx/PEI/SWNT film)₂/Pt. Meanwhile, PDMS is applied onto a SU-8-based fluidic channel master (1.5 mm (L)×2.5 mm (W)×200 um (T)) formed on a silicon wafer by photolithography, and then heated so as to form a PDMS cover. An inlet and an outlet having a diameter of about 0.5 mm are formed at both ends of the channel. The PDMS fluidic cover thus fabricated is stacked on the double-layered PDMS film having the working electrode and the reference electrode and the counter electrode, respectively so as to fabricate a transparent flexible microfluidic glucose sensor. Further, to facilitate capillary action of an analyte in the channel, the SU-8 substrate may be treated using a hydrophobic material.

FIG. 15 is a schematic illustration showing a fabrication process of a transparent flexible multi-layered hybrid electronic sheet-GOx-based biosensor according to a specific embodiment.

2.4. Comparison of Electrochemical Property of Single-Layered Hybrid Electronic Sheet-GOx-Based Biosensor 1

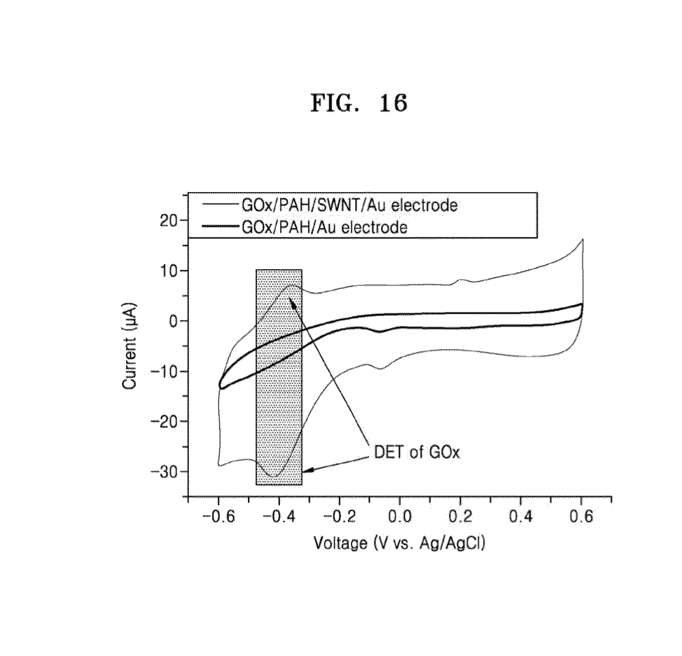

The electrode prepared in Example 2.1.1 and the electrode of Comparative Example prepared in Example 2.1.3 are used as working electrodes, the Pt-coated titanium chamber is used as a counter electrode, and Ag/AgCl (3M KCl saturated, PAR, K0260) is used as a reference electrode (RE), and 10 mM PBS buffer (pH=7.4, 79383, Sigma Aldrich) is used as an electrolyte to perform cyclic voltammetry (CV). To observe DET of GOx, the measurement voltage is applied in a range of −0.6 V˜0.6 V versus the Ag/AgCl reference electrode at a scan rate of 200 mV/s. The result is shown in FIG. 16.

FIG. 16 is a CV graph showing a comparison of direct electron transfer (DET) reaction between the single-layered hybrid electronic sheet-GOx based biosensor according to an exemplary embodiment and the GOx electrode formed on the gold electrode without the hybrid electronic sheet.

As shown in FIG. 16, the single-layered hybrid electronic sheet—transferred GOx electrode according to an exemplary embodiment shows strong redox peaks in the region of −0.4 V, whereas the non-hybrid electronic sheet-transferred electrode shows no redox peaks in the region of −0.4 V. This result indicates that the hybrid electronic sheet has high electrochemical activity and also effectively causes direct electron transfer (DET) with GOx in the closer region, compared to the gold electrode without the hybrid electronic sheet. In particular, because the FAD redox center of GOx is buried inside a thick protein layer, the electrode should be located as close as 1-2 nm or less for effective DET. When the hybrid electronic sheet is used, GOx is effectively immobilized on the hybrid electronic sheet in close enough proximity to allow DET by a simple layer-by-layer method. Further, a high capacitive current is observed in the voltage range of −0.6 V˜0.6 V, compared to the electrode of Comparative Example. These results indicate that the hybrid electronic sheet has a higher electrochemical reactivity and a larger surface area than the electrode of Comparative Example.

2.5. Comparison of Electrochemical Property of Single-Layered Hybrid Electronic Sheet-GOx-Based Biosensor 2

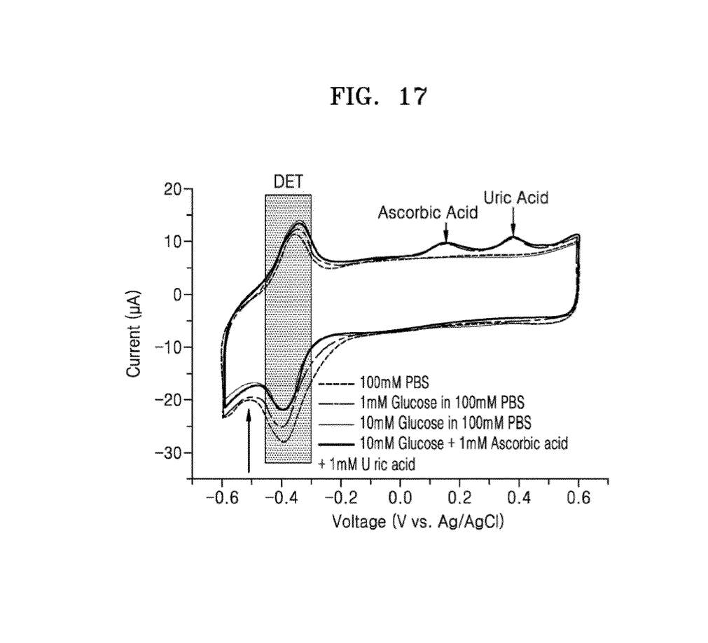

To evaluate whether the electrode prepared in Example 2.1.1 is used effectively to screen ascorbic acid and uric acid, a 3-electrode system as mentioned in Example 2.4 is used. 10 mM glucose (G7528, Sigma Aldrich) in 10 mM PBS buffer (pH=7.4, 79383, Sigma Aldrich) as an electrolyte is prepared, and 1 mM ascorbic acid (A5963, Sigma Aldrich) and uric acid (U0881, Sigma Aldrich are mixed with the solution of the same concentration (10 mM glucose in 10 mM PBS buffer) to prepare a comparative solution. To observe DET of GOx, the measurement voltage is applied in a range of −0.6 V˜0.6 V versus the Ag/AgCl reference electrode at a scan rate of 200 mV/s. The result is shown in FIG. 17.

FIG. 17 is a graph showing a comparison between a current response to glucose and a current response to a mixture of glucose with ascorbic acid and uric acid in the single-layered hybrid electronic sheet-GOx based biosensor according to a specific embodiment.

As shown in FIG. 17, when 1 mM or 10 mM glucose solution is injected as the electrolyte, a reduction current is reduced, compared to use of 10 mM PBS (0 mM Glucose). This result indicates that the equilibrium reaction of FAD-FADH2 of GOx on the electrode proceeds at 0 mM glucose, and FAD is converted to FADH2 by enzymatic reaction due to addition of glucose to the electrolyte, leading to reduction of FAD which is consumed by a reduction reaction on the electrode, and thus the reduction current by addition of glucose is reduced, compared to the reduction current at 0 mM glucose. Further, when the results are compared between addition of 10 mM glucose and addition of 1 mM ascorbic acid or uric acid with 10 mM glucose, two oxidation currents are detected by oxidation of ascorbic acid and uric acid in the range of 0.2 V and 0.4 V, whereas there is no difference between addition of ascorbic acid and uric acid and addition of pure 10 mM glucose in the DET range. These results indicate that DET reaction of GOx detected at the negative voltage (−0.4 V) is not influenced by ascorbic acid or uric acid which is the interfering factor causing electrochemical reaction at the positive voltage (0.2 V or 0.4 V), and thus selectivity for glucose is high.

2.6. Comparison of Electrochemical Property of Single-Layered Hybrid Electronic Sheet-GOx-Based Biosensor 3

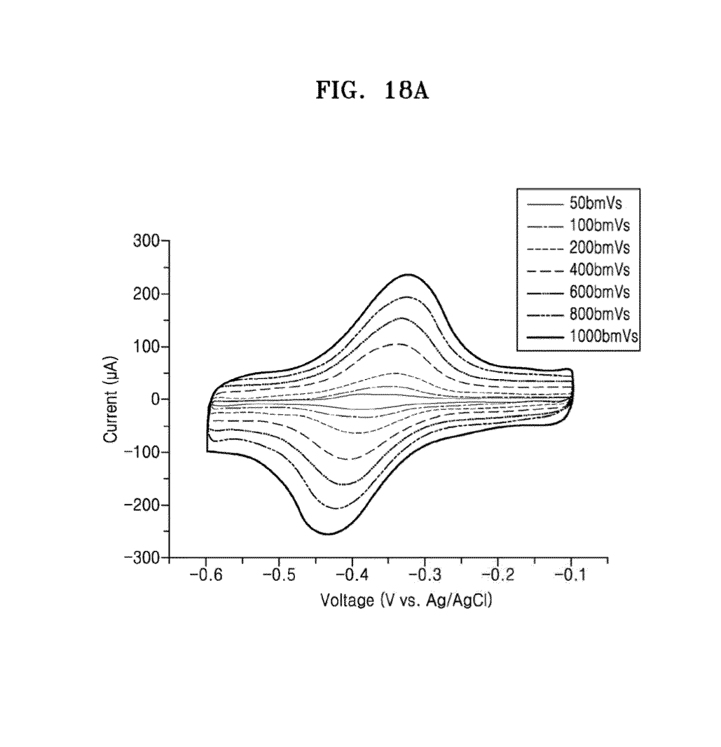

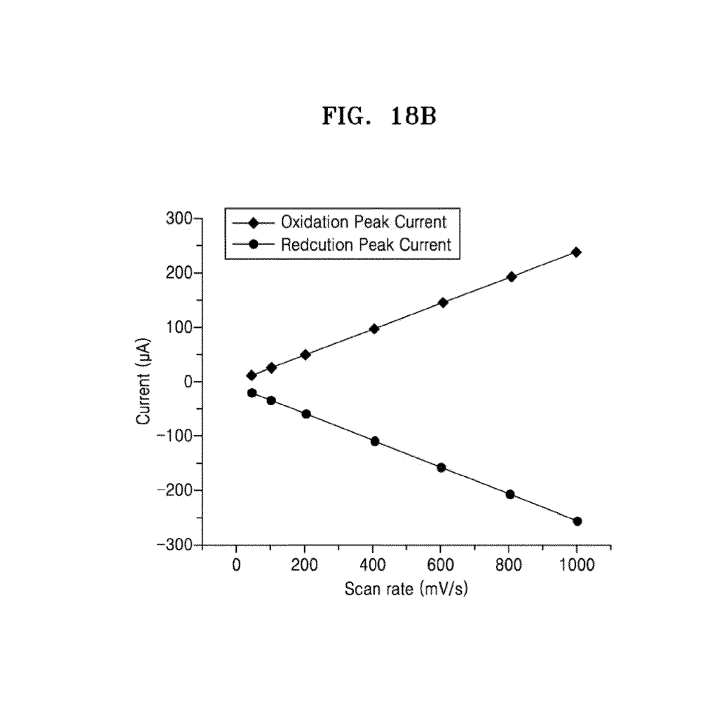

To examine whether DET reaction of GOx on the electrode prepared in Example 2.1.1 is effectively immobilized and adsorbed onto the electrode, a 3-electrode system as mentioned in Example 2.4 is used. 10 mM PBS buffer (pH=7.4, 79383, Sigma Aldrich) is used as an electrolyte, and properties of the electrode are examined at a variety of scan rates (50, 100, 200, 400, 600, 800 and 1000 mV/s). To measure only pure DET of GOx, the measurement voltage is limited to a range of −0.1 V˜0.6 V versus the Ag/AgCl reference electrode. The result is shown in FIG. 18.

FIGS. 18A and 18B are graphs showing changes in pure DET current response according to a voltage scan rate in the single-layered hybrid electronic sheet-GOx based biosensor according to a specific embodiment.

As shown in FIG. 18a, at 0 mM glucose, that is, at the equilibrium of FAD-FADH2 of GOx of the electrode, the redox current of DET increases by varying the scan rate. As shown in FIG. 18b, both oxidation and reduction currents are linearly proportional to the scan rate (R2-0.99). These results indicate that GOx is immobilized/adsorbed onto the hybrid electronic sheet in close proximity.

2.7. Test of Sensitivity of Single-Layered Hybrid Electronic Sheet-GOx-Based Biosensor

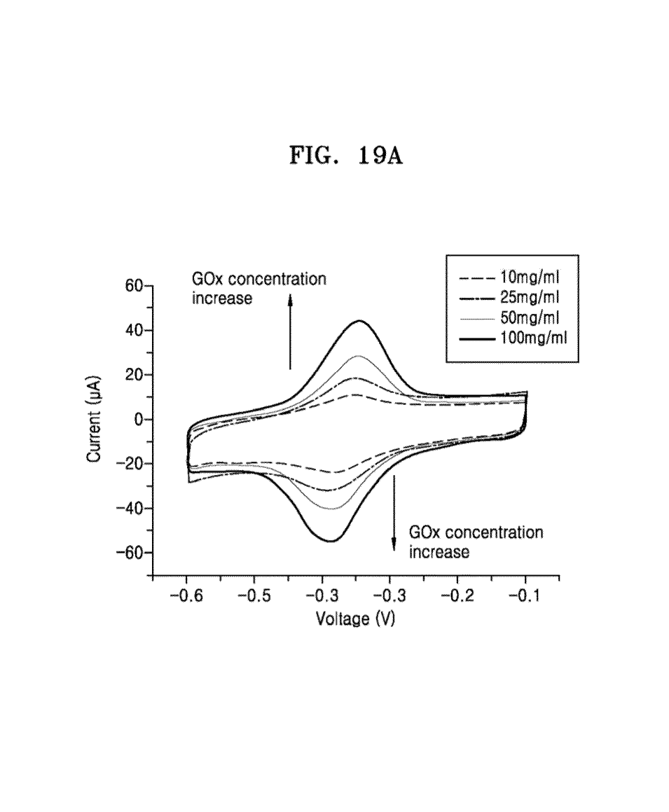

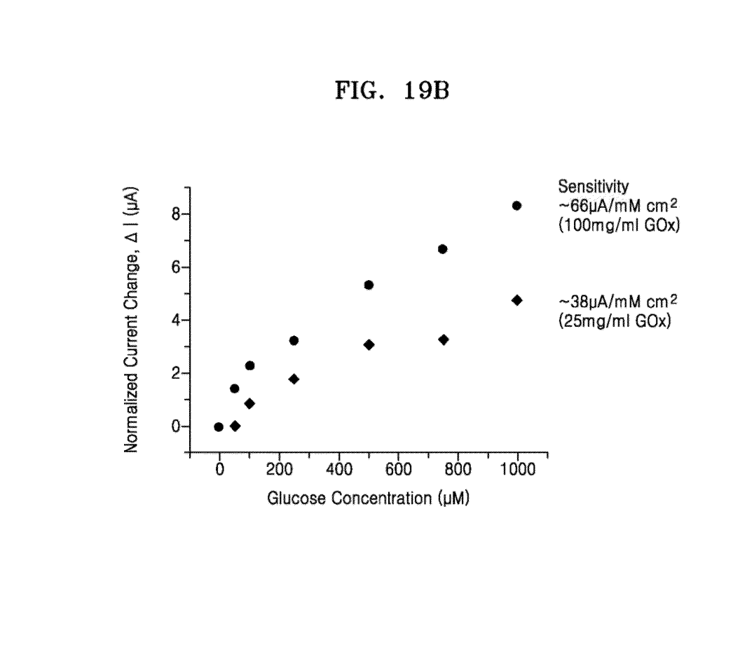

To evaluate properties of 4 electrodes prepared in Example 2.1.2, a 3-electrode system as mentioned in Example 2.4 is used. 10 mM PBS buffer (pH=7.4, 79383, Sigma Aldrich) is used as an electrolyte, and properties of the electrode are examined at a scan rate of 200 mV/s. To measure only pure DET of GOx, the measurement voltage is limited to a range of −0.1 V˜−0.6 V versus the Ag/AgCl reference electrode. The result is shown in FIG. 19.

FIG. 19A is a graph showing that DET redox of GOx increases with increasing concentration of immobilized GOx when different concentrations of GOx are immobilized on the hybrid electronic sheet according to a specific embodiment.

FIG. 19B is a graph showing that a current response to a change in glucose concentration increases with increasing concentration of GOx immobilized on the hybrid electronic sheet according to a specific embodiment, leading to an increase in sensor sensitivity.

As shown in FIG. 19A, at 0 mM glucose, that is, at the equilibrium of FAD-FADH2 of GOx of the electrode, the DET redox current of GOx immobilized on the hybrid electronic sheet increases with increasing GOx integration, when 4 electrodes are examined under the same conditions.

As shown in FIG. 19B, when glucose is added by varying its concentration (0.1, 0.25, 0.5, 0.75, 1 mM) to the electrode immobilized with 25 mg/mL GOx or 100 mg/mL GOx, the electrode immobilized with high concentration of GOx (100 mg/mL) shows sensitivity of about 66 uA/mM cm², whereas the electrode immobilized with low concentration of GOx (25 mg/mL) shows sensitivity of about 38 uA/mM cm², indicating that high concentration of GOx is immobilized on the hybrid electronic sheet having a large surface area prepared by using SWNT-based nanomaterials so as to increase sensitivity of the sensor.

2.8. Test of Sensitivity of Multi-Layered Hybrid Electronic Sheet-GOx-Based Biosensor

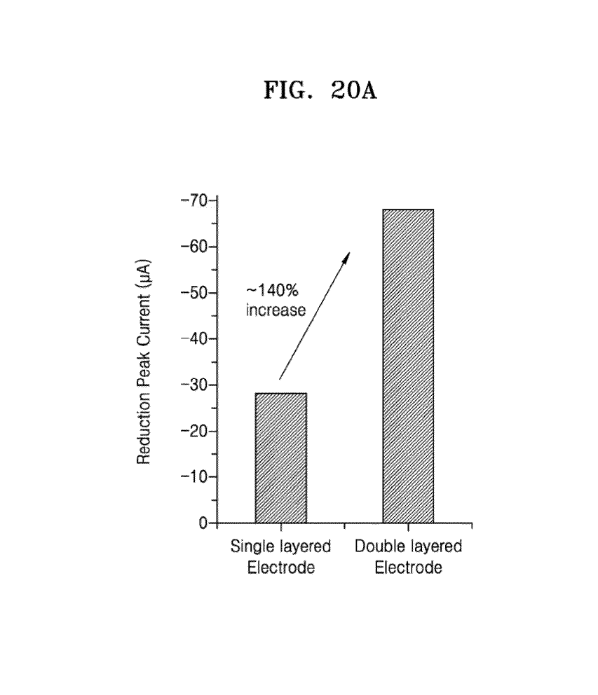

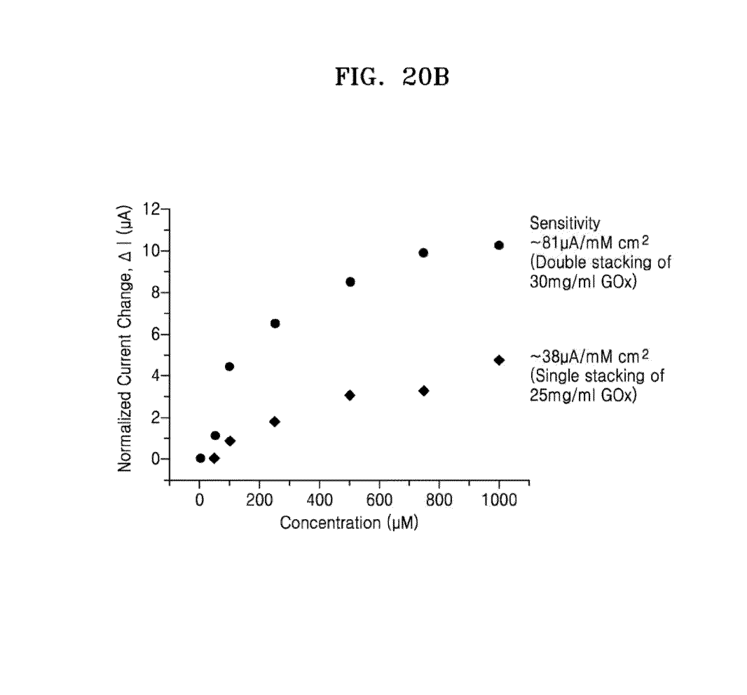

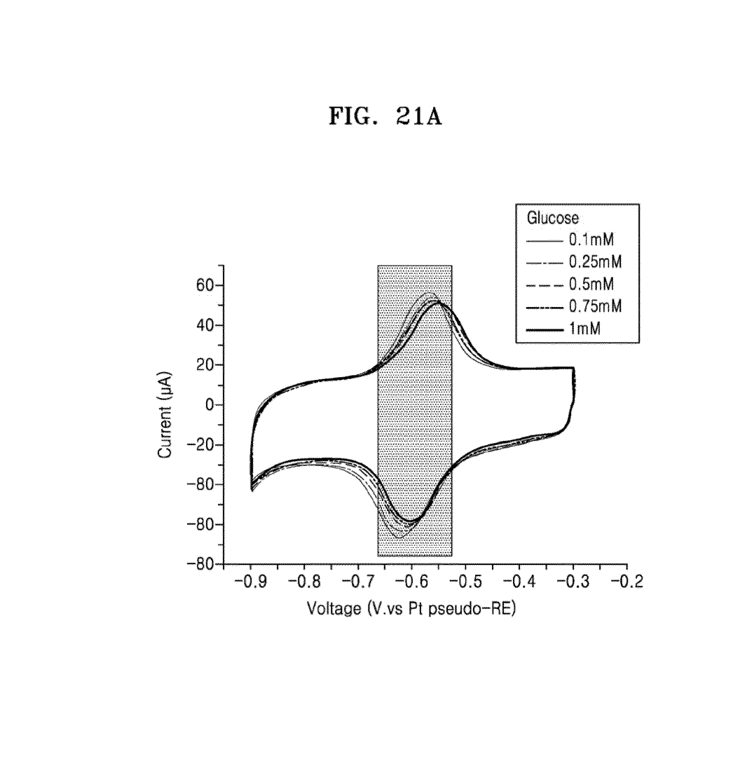

To evaluate sensitivity of the biosensor fabricated in Example 2.2, a 3-electrode system as mentioned in Example 2.4 is used. 10 mM PBS buffer (pH=7.4, 79383, Sigma Aldrich) is used as an electrolyte, and property of the electrode is examined at a scan rate of 200 mV/s. To measure only pure DET of GOx, the measurement voltage is limited to a range of −0.1 V˜−0.6 V versus the Ag/AgCl reference electrode. The result is shown in FIG. 20.

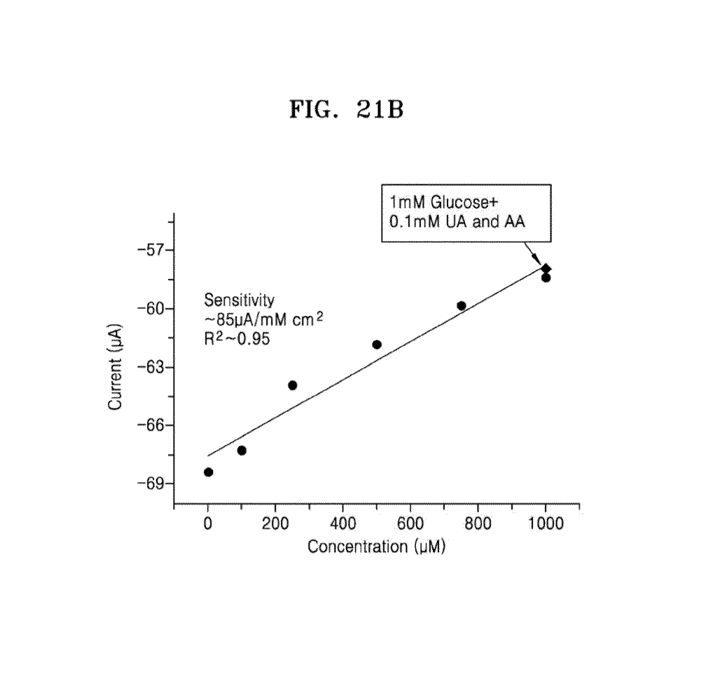

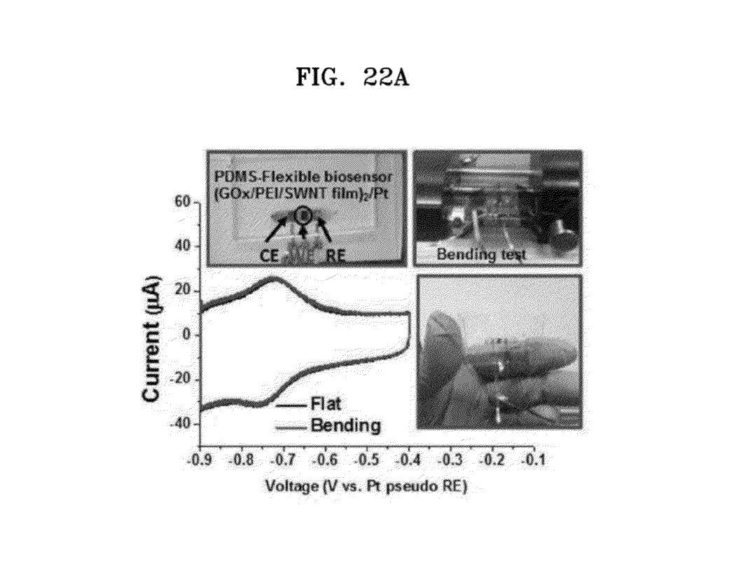

To examine applicability to the human body, 13 mm²-Pt on SPE 250BT is used as a biocompatible pseudo-Pt reference electrode, instead of Ag/AgCl, and property of the electrode is examined in the same manner as above. The result is shown in FIG. 21.

FIG. 20A is a graph showing that a high DET reduction current increases in the multi-layered hybrid electronic sheet-GOx-based biosensor according to a specific embodiment, compared to the single-layered structure.

FIG. 20B is a graph showing that sensor sensitivity may be increased by multi-stacking, in which sensitivity to a change in glucose concentration increases in the multi-layered hybrid electronic sheet-GOx-based biosensor according to a specific embodiment, compared to the single-layered structure.

As shown in FIG. 20A, at 0 mM glucose, the multi-layered structure shows about 140% increase in DET reaction of GOx, compared to the single-layered structure.

As shown in FIG. 20B, at 0.1, 0.25, 0.5, 0.75, or 1 mM glucose, the multi-layered biosensor shows sensitivity of about 81 uA/mM cm², whereas the single-layered biosensor shows sensitivity of about 38 uA/mM cm², indicating an about 100% increase. These results indicate that the multi-layered hybrid electronic sheet-GOx-based biosensor according to an exemplary embodiment is able to detect glucose in the body fluid having low glucose levels, such as tear, with high sensitivity.

FIGS. 21A and 21B are graphs showing results of measuring sensitivity of the multi-layered hybrid electronic sheet-GOx-based biosensor according to an exemplary embodiment in a reference electrode harmless to human body.

As shown in FIG. 21A, as the glucose concentration increases, the value of reduction current is gradually reduced. As shown in FIG. 21B, the sensitivity is about 85 uA/mM cm², which is almost similar to 81 uA/mM cm² measured when Ag/AgCl is used. When 1 mM glucose is added together with 0.1 mM ascorbic acid or uric acid, the error value is 1% or less, compared to addition of 1 mM glucose only. Therefore, it can be seen that DET reaction of the multi-layered hybrid electronic sheet-GOx-based biosensor according to an exemplary embodiment effectively occurs without interference of ascorbic acid and uric acid, even though the pseudo-Pt reference electrode is used.

2.9. Test of Sensitivity and Flexibility of Transparent Flexible Multi-Layered Hybrid Electronic Sheet-GOx-Based Biosensor

To evaluate sensitivity of the transparent flexible multi-layered hybrid electronic sheet-GOx-based biosensor fabricated in Example 2.3, a 3-electrode system as mentioned in Example 2.4 is used. 10 mM PBS buffer (pH=7.4, 79383, Sigma Aldrich) is used as an electrolyte, and property of the electrode is examined at a scan rate of 200 mV/s. To measure only pure DET of GOx, the measurement voltage is limited to a range of −0.9 V˜-0.2 V versus the pseudo-Pt reference electrode.

Further, to evaluate flexibility of the biosensor, the biosensor is placed on a polyimide film, and then a syringe pump is used to bend it at an angle of about 50° and to measure CV, which is compared with CV before bending.

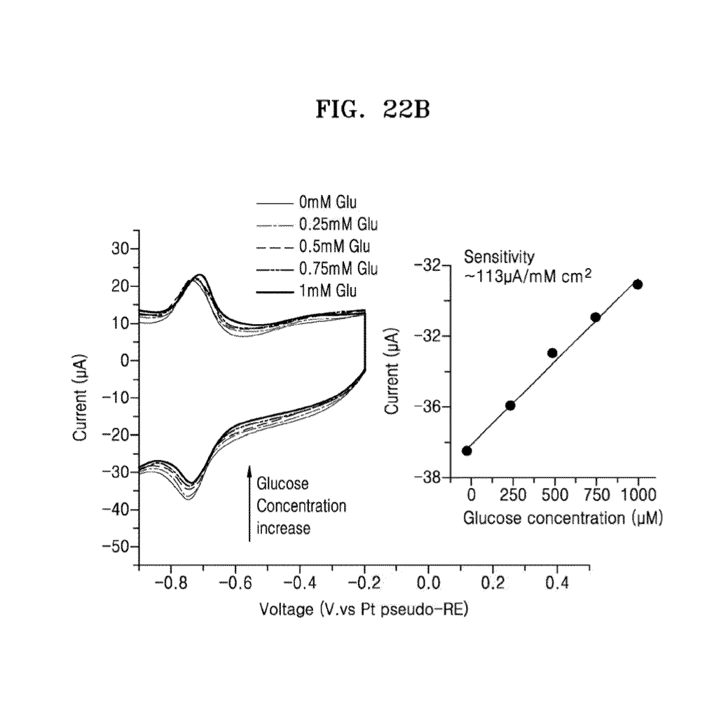

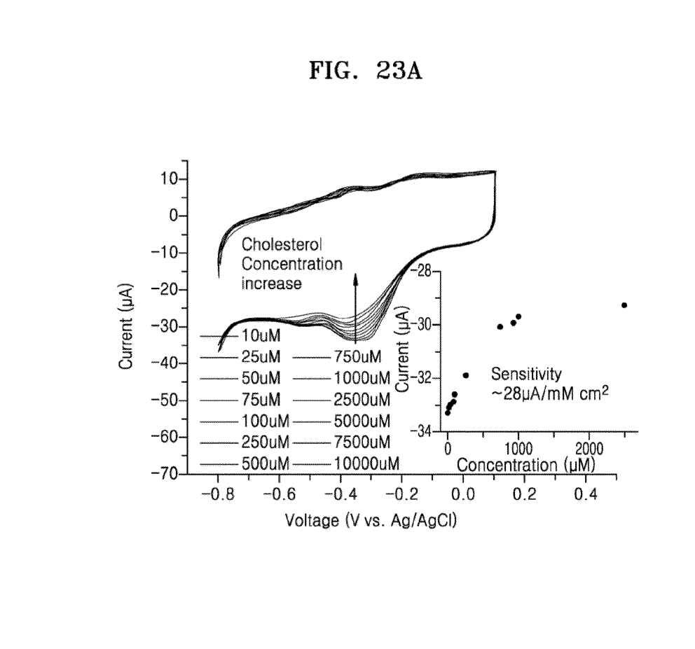

The result is shown in FIG. 22.

FIGS. 22A and 22B are graphs showing sensitivity and flexibility of the transparent flexible multi-layered hybrid electronic sheet-GOx-based biosensor according to a specific embodiment.

As shown in FIG. 22A, there is no difference in CV between the biosensor bent at the angle of about 50° and the flat biosensor, indicating that the transparent flexible multi-layered hybrid electronic sheet-GOx-based biosensor according to an exemplary embodiment can be used as a wearable device.

As shown in FIG. 22B, when glucose is added in the range of 0.1, 0.25, 0.5, 0.75, or 1 mM, the transparent flexible multi-layered hybrid electronic sheet-GOx-based biosensor according to an exemplary embodiment shows a linear decrease in the reduction current under microfluidic system environment, and the sensitivity of the electrode is about 113 uA/mM cm². These results indicate that the transparent flexible multi-layered hybrid electronic sheet-GOx-based biosensor according to an exemplary embodiment is used as a flexible device to detect glucose in the body fluid having low glucose levels, such as tear, with high sensitivity.

3. Fabrication and Characterization of Hybrid Electronic Sheet-Cholesterol Oxidase or Lactate Oxidase-Based Biosensor

3.1. Fabrication of Hybrid Electronic Sheet-Cholesterol Oxidase or Lactate Oxidase-Based Biosensor

A biosensor is fabricated in the same manner as in 2.1.1, except that 5 μl of 10 mg/ml cholesterol oxidase (CholOx) or 50 mg/ml lactate oxidase (LOx) is mixed with 100 mM PBS buffer solution, and the mixture is immobilized on a positively charged electrode.

3.2. Test of Sensitivity of Hybrid Electronic Sheet-Cholesterol Oxidase or Lactate Oxidase-Based Biosensor

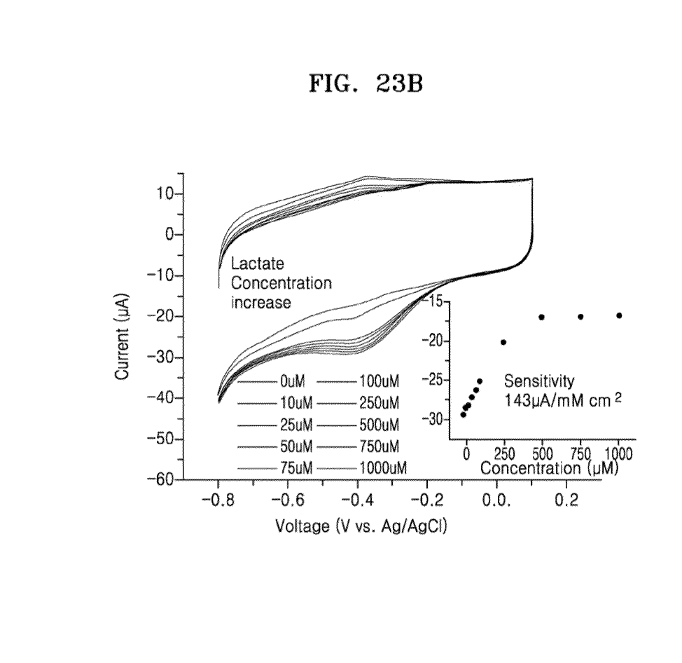

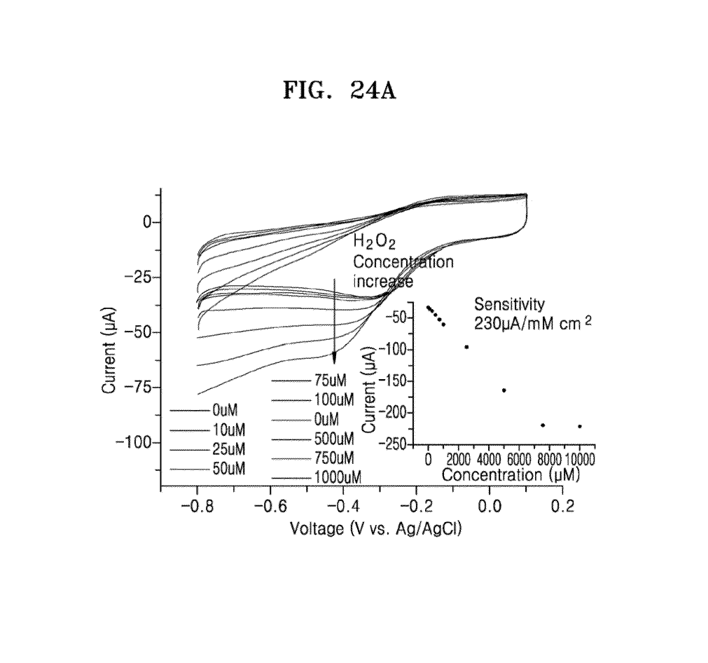

To evaluate sensitivity of the hybrid electronic sheet-cholesterol oxidase or lactate oxidase-based biosensor, a 3-electrode system as mentioned in Example 2.4 is used. 10 mM PBS buffer (pH=7.4, 79383, Sigma Aldrich) is used as an electrolyte, and property of the electrode is examined at a scan rate of 200 mV/s. To measure only pure DET of the FAD-based enzymes, the measurement voltage is limited to a range of −0.1 V˜-0.6 V versus the Ag/AgCl reference electrode. The result is shown in FIG. 23.

FIG. 23A is a graph showing sensitivity of a hybrid electronic sheet-cholesterol oxidase-based biosensor according to a specific embodiment.

FIG. 23B is a graph showing sensitivity of a hybrid electronic sheet-lactate oxidase-based biosensor according to a specific embodiment.

As shown in FIG. 23A, when cholesterol is added in the range of 0.01, 0.025, 0.05, 0.075, 0.1, 0.25, 0.5, 0.75, 1, or 2.5 mM, the reduction current decreases at −0.4 V versus the reference electrode, with increasing cholesterol concentration, and the sensitivity of the electrode is about 28 uA/mM cm² at 0-1 mM.

As shown in FIG. 23B, when L-lactate is added in the range of 0.01, 0.025, 0.05, 0.075, 0.1, 0.25, 0.5, 0.75, 1, or 2.5 mM, the reduction current decreases, and the sensitivity of the electrode is about 143 uA/mM cm² at 0-0.5 mM.

4. Fabrication and characterization of hybrid electronic sheet-HRP or catalase-based biosensor

4.1. Fabrication of Hybrid Electronic Sheet-HRP or Catalase-Based Biosensor

A hybrid electronic sheet-catalase-based biosensor is fabricated in the same manner as in 2.1.1, except that 5 μl of 10 mg/ml catalase is mixed with 100 mM PBS buffer solution, and the mixture is immobilized onto a positively charged electrode.

Further, a hybrid electronic sheet-HRP-based biosensor is fabricated in the same manner as in 2.1.1, except that the surface of the PAH-coated electrode is modified to be negatively charged using 5 μl of 6 mg/ml polystyrene sulfonate (PSS), and 5 μl of 10 mg/ml HRP is mixed with 100 mM PBS buffer solution and the mixture is immobilized on the negatively charged electrode.

4.2. Test of Sensitivity of Hybrid Electronic Sheet-HRP or Catalase-Based Biosensor

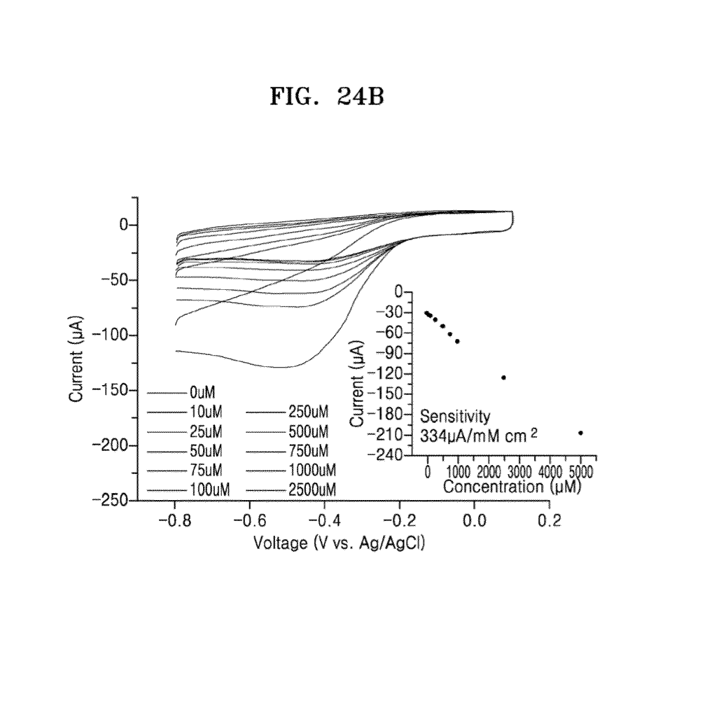

To evaluate sensitivity of the hybrid electronic sheet-HRP or catalase-based biosensor, a 3-electrode system as mentioned in Example 2.4 is used. 10 mM PBS buffer (pH=7.4, 79383, Sigma Aldrich) is used as an electrolyte, and property of the electrode is examined at a scan rate of 200 mV/s. To measure only pure Heme-based DET, the measurement voltage is limited to a range of −0.1 V˜-0.6 V versus the Ag/AgCl reference electrode. The result is shown in FIG. 24.

FIG. 24A is a graph showing sensitivity of the hybrid electronic sheet-HRP-based biosensor according to a specific embodiment.

FIG. 24B is a graph showing sensitivity of the hybrid electronic sheet-catalase-based biosensor according to a specific embodiment.

As shown in FIG. 24A, when H₂O₂ is added in the range of 0.01, 0.025, 0.05, 0.075, 0.1, 0.25, 0.5, 0.75, 1, or 2.5 mM, the reduction current increases at −0.4 V versus the reference electrode, with increasing H₂O₂ concentration, and the sensitivity of the electrode is about 230 uA/mM cm² at 0-5 mM.

As shown in FIG. 24B, when H₂O₂ is added in the range of 0.01, 0.025, 0.05, 0.075, 0.1, 0.25, 0.5, 0.75, 1, or 2.5 mM, the reduction current increases at −0.4 V versus the reference electrode, with increasing H₂O₂ concentration, and the sensitivity of the electrode is about 334 uA/mM cm² at 0-5 mM.

These results indicate that when the H₂O₂ concentration increases, Heme redox center in the enzyme is oxidized by H₂O₂, and the increased oxidized Heme is reduced again by electrochemical reaction, leading to the increase in the reduction current.

5. Fabrication and Characterization of Hybrid Electronic Sheet-Laccase, Tyrosinase or Galactose Oxidase-Based Biosensor

5.1. Fabrication of Hybrid Electronic Sheet-Laccase, Tyrosinase or Galactose Oxidase-Based Biosensor

A hybrid electronic sheet-laccase or tyrosinase-based biosensor is fabricated in the same manner as in 2.1.1, except that 5 μl of 50 mg/ml laccase or 20 mg/ml tyrosinase is mixed with 100 mM PBS buffer solution, and the mixture is immobilized onto a positively charged electrode.

Further, a hybrid electronic sheet-GalOx-based biosensor is fabricated in the same manner as in 2.1.1, except that the surface of the PAH-coated electrode is modified to be negatively charged using 5 μl of 6 mg/ml polystyrene sulfonate (PSS), and 5 μl of 3 mg/ml galactose oxidase (GalOx) is mixed with 100 mM PBS buffer solution, and the mixture is immobilized on the negatively charged electrode.

5.2. Test of Sensitivity of Hybrid Electronic Sheet-Laccase, Tyrosinase or Galactose Oxidase-Based Biosensor

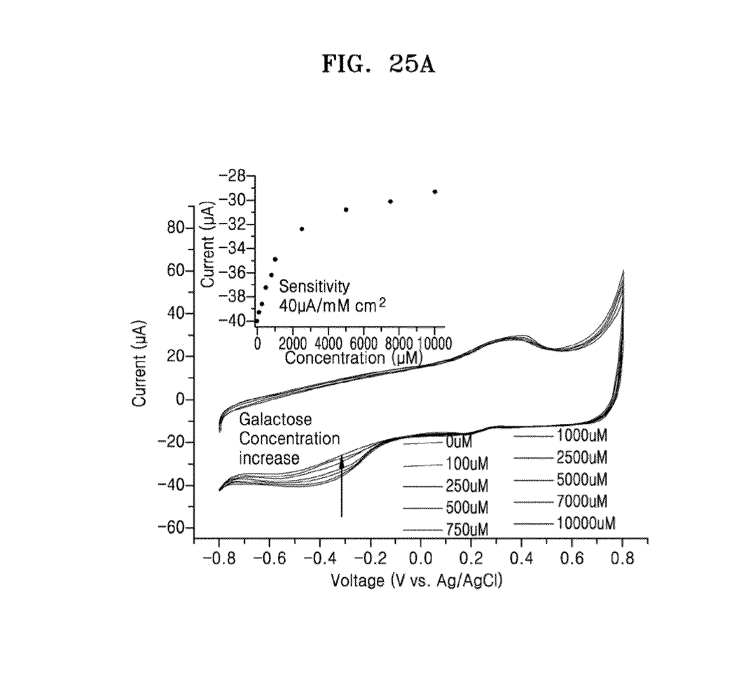

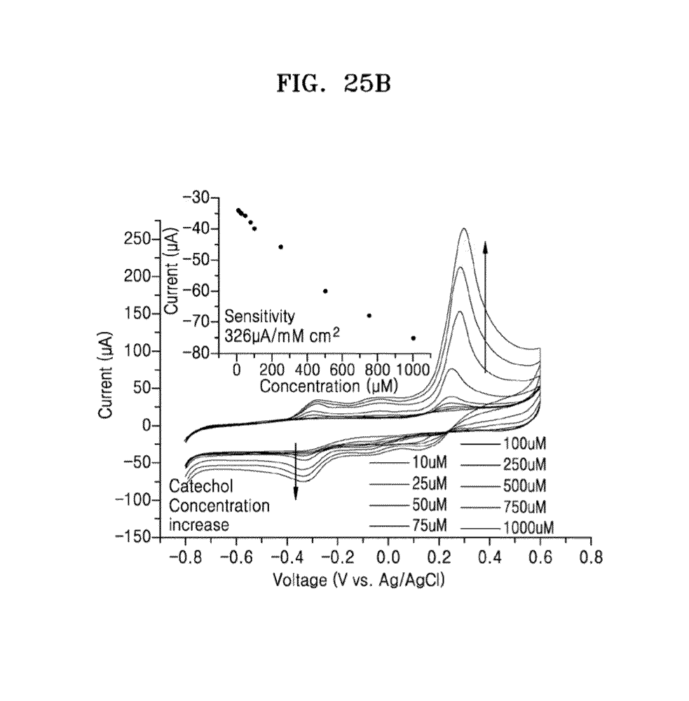

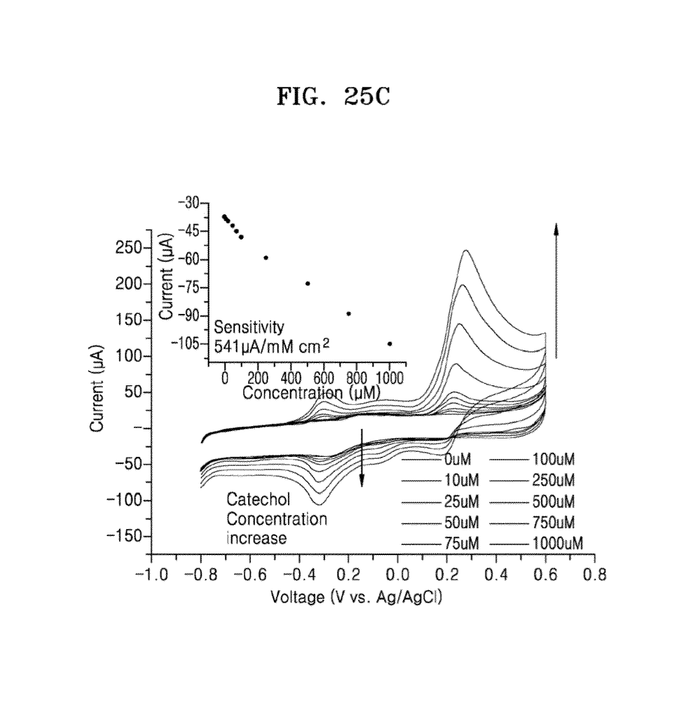

To evaluate sensitivity of the hybrid electronic sheet-laccase, tyrosinase or galactose oxidase-based biosensor, a 3-electrode system as mentioned in Example 2.4 is used. 10 mM PBS buffer (pH=7.4, 79383, Sigma Aldrich) is used as an electrolyte, and property of the electrode is examined at a scan rate of 200 mV/s. The result is shown in FIG. 25.

FIG. 25A is a graph showing sensitivity of the hybrid electronic sheet-galactose oxidase-based biosensor according to a specific embodiment.

FIG. 25B is a graph showing sensitivity of the hybrid electronic sheet-tyrosinase-based biosensor according to a specific embodiment.

FIG. 25C is a graph showing sensitivity of the hybrid electronic sheet-laccase-based biosensor according to a specific embodiment.

As shown in FIG. 25A, when galactose is added in the range of 0.01, 0.025, 0.05, 0.075, 0.1, 0.25, 0.5, 0.75, 1, or 2.5 mM, the reduction current decreases at −0.4 V versus the reference electrode, with increasing galactose concentration, and the sensitivity of the electrode is about 40 uA/mM cm² at 0-1 mM.

As shown in FIG. 25B, when catechol is added in the range of 0.01, 0.025, 0.05, 0.075, 0.1, 0.25, 0.5, 0.75, 1, or 2.5 mM, the reduction current increases at −0.35 V versus the reference electrode, with increasing catechol concentration, and the sensitivity of the electrode is about 326 uA/mM cm² at 0-1 mM.

As shown in FIG. 25C, when catechol is added in the range of 0.01, 0.025, 0.05, 0.075, 0.1, 0.25, 0.5, 0.75, 1, or 2.5 mM, the reduction current increases at −0.35 V versus the reference electrode, with increasing catechol concentration, and the sensitivity of the electrode is about 541 uA/mM cm² at 0-1 mM.

A biosensor according to an aspect has high electrochemical activity and allows DET-based detection of an analyte in a sample.

A wearable device according to an aspect has high sensitivity and selectivity to an analyte while being harmless to the human body, and thus allows non-invasive detection of a small amount of analyte in a sample.

It should be understood that exemplary embodiments described herein should be considered in a descriptive sense only and not for purposes of limitation. Descriptions of features or aspects within each exemplary embodiment should typically be considered as available for other similar features or aspects in other exemplary embodiments.

While one or more exemplary embodiments have been described with reference to the figures, it will be understood by those of ordinary skill in the art that various changes in form and details may be made therein without departing from the spirit and scope as defined by the following claims.

What is claimed is:

1. A biosensor comprising:
a substrate;
an electronic sheet formed on the substrate; and
an analyte-binding material immobilized on the electronic sheet,
wherein the electronic sheet comprises a graphitic material and a phage binding to the graphitic material, and the binding of the graphitic material and the phage occurs between the graphitic material and a peptide displayed on a coat protein of the phage or a fragment thereof.

2. The biosensor of claim 1, wherein the substrate is an insulating substrate and one or more electrodes are disposed on the substrate.

3. The biosensor of claim 2, wherein the one or more electrodes comprise a first electrode and a second electrode.

4. The biosensor of claim 3, wherein the electronic sheet is disposed on the first electrode or on a portion thereof.

5. The biosensor of claim 1, wherein the substrate is a transparent flexible substrate.

6. The biosensor of claim 1, wherein the electronic sheet is patterned on the substrate.

7. The biosensor of claim 1, wherein the graphitic material comprises one or more selected from the group consisting of a graphene sheet, a highly ordered pyrolytic graphite (HOPG) sheet, a single-walled carbon nanotube, a double-walled carbon nanotube, a multi-walled carbon nanotube, and fullerene.

8. The biosensor of claim 1, wherein the graphitic material comprises a graphene sheet and a single-walled carbon nanotube.

9. The biosensor of claim 1, wherein the peptide comprises one or more selected from the group consisting of amino acid sequences of SEQ ID NOS. 1 to 8.

10. The biosensor of claim 1, wherein the phage is M13 phage, F1 phage, Fd phage, If1 phage, Ike phage, Zj/Z phage, Ff phage, Xf phage, Pf1 phage or Pf3 phage.

11. The biosensor of claim 1, wherein the analyte-binding material is oxidase, peroxidase, reductase, catalase or dehydrogenase.

12. The biosensor of claim 1, further comprising a protection layer formed on the immobilized analyte-binding material.

13. The biosensor of claim 1, wherein the electronic sheet has a surface contacting the analyte-binding material, and a surface of the electronic sheet has a positive or negative charge that is opposite to a charge of the analyte-binding material.

14. The biosensor of claim 1, wherein the biosensor comprises a plurality of repeating units, each repeating unit comprising the electronic sheet and the analyte-binding material.

15. The biosensor of claim 1, further comprising a test cell for accommodating a sample, the electronic sheet, and the analyte-binding material, wherein the test cell has a channel having an inlet for accepting the sample and an outlet for discharging the sample.

16. A wearable device for detecting bioinformation, comprising the biosensor of claim 1.

17. The wearable device of claim 15, wherein the wearable device is a contact lens.


SEQUENCE LISTING (14 sequences)

SEQ ID NO: 1 - protein, 8 aa, Artificial Sequence
  peptide selectively binding to graphitic materials
XSXAAXXP

SEQ ID NO: 2 - protein, 8 aa, Artificial Sequence
  peptide selectively binding to graphitic materials
XXPXXAXP

SEQ ID NO: 3 - protein, 7 aa, Artificial Sequence
  peptide selectively binding to graphitic materials
SXAAXXP

SEQ ID NO: 4 - protein, 7 aa, Artificial Sequence
  peptide selectively binding to graphitic materials
XPXXAXP

SEQ ID NO: 5 - protein, 8 aa, Artificial Sequence
  peptide selectively binding to graphitic materials
DSWAADIP

SEQ ID NO: 6 - protein, 8 aa, Artificial Sequence
  peptide selectively binding to graphitic materials
DNPIQAVP

SEQ ID NO: 7 - protein, 7 aa, Artificial Sequence
  peptide selectively binding to graphitic materials
SWAADIP

SEQ ID NO: 8 - protein, 7 aa, Artificial Sequence
  peptide selectively binding to graphitic materials
NPIQAVP

SEQ ID NO: 9 - DNA, 7222 bp, Artificial Sequence
  cloning vector M13KE
aatgctactactattagtagaattgatgccaccttttcagctcgcgccccaaatgaaaatatagctaaacaggttattgaccatttgcgaaatgtatctaatggtcaaactaaatctactcgttcgcagaattgggaatcaactgttatatggaatgaaacttccagacaccgtactttagttgcatatttaaaacatgttgagctacagcattatattcagcaattaagctctaagccatccgcaaaaatgacctcttatcaaaaggagcaattaaaggtactctctaatcctgacctgttggagtttgcttccggtctggttcgctttgaagctcgaattaaaacgcgatatttgaagtctttcgggcttcctcttaatctttttgatgcaatccgctttgcttctgactataatagtcagggtaaagacctgatttttgatttatggtcattctcgttttctgaactgtttaaagcatttgagggggattcaatgaatatttatgacgattccgcagtattggacgctatccagtctaaacattttactattaccccctctggcaaaacttcttttgcaaaagcctctcgctattttggtttttatcgtcgtctggtaaacgagggttatgatagtgttgctcttactatgcctcgtaattccttttggcgttatgtatctgcattagttgaatgtggtattcctaaatctcaactgatgaatctttctacctgtaataatgttgttccgttagttcgttttattaacgtagatttttcttcccaacgtcctgactggtataatgagccagttcttaaaatcgcataaggtaattcacaatgattaaagttgaaattaaaccatctcaagcccaatttactactcgttctggtgtttctcgtcagggcaagccttattcactgaatgagcagctttgttacgttgatttgggtaatgaatatccggttcttgtcaagattactcttgatgaaggtcagccagcctatgcgcctggtctgtacaccgttcatctgtcctctttcaaagttggtcagttcggttcccttatgattgaccgtctgcgcctcgttccggctaagtaacatggagcaggtcgcggatttcgacacaatttatcaggcgatgatacaaatctccgttgtactttgtttcgcgcttggtataatcgctgggggtcaaagatgagtgttttagtgtattcttttgcctctttcgttttaggttggtgccttcgtagtggcattacgtattttacccgtttaatggaaacttcctcatgaaaaagtctttagtcctcaaagcctctgtagccgttgctaccctcgttccgatgctgtctttcgctgctgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctgataaaccgatacaattaaaggctccttttggagccttttttttggagattttcaacgtgaaaaaattattattcgcaattcctttagtggtacctttctattctcactcggccgaaactgttgaaagttgtttagcaaaatcccatacagaaaattcatttactaacgtctggaaagacgacaaaactttagatcgttacgctaactatgagggctgtctgtggaatgctacaggcgttgtagtttgtactggtgacgaaactcagtgttacggtacatgggttcctattgggcttgctatccctgaaaatgagggtggtggctctgagggtggcggttctgagggtggcggttctgagggtggcggtactaaacctcctgagtacggtgatacacctattccgggctatacttatatcaaccctctcgacggcacttatccgcctggtactgagcaaaaccccgctaatcctaatccttctcttgaggagtctcagcctcttaatactttcatgtttcagaataataggttccgaaataggcagggggcattaactgtttatacgggcactgttactcaaggcactgaccccgttaaaacttattaccagtacactcctgtatcatcaaaagccatgtatgacgcttactggaacggtaaattcagagactgcgctttccattctggctttaatgaggatttatttgtttgtgaatatcaaggccaatcgtctgacctgcctcaacctcctgtcaatgctggcggcggctctggtggtggttctggtggcggctctgagggtggtggctctgagggtggcggttctgagggtggcggctctgagggaggcggttccggtggtggctctggttccggtgattttgattatgaaaagatggcaaacgctaataagggggctatgaccgaaaatgccgatgaaaacgcgctacagtctgacgctaaaggcaaacttgattctgtcgctactgattacggtgctgctatcgatggtttcattggtgacgtttccggccttgctaatggtaatggtgctactggtgattttgctggctctaattcccaaatggctcaagtcggtgacggtgataattcacctttaatgaataatttccgtcaatatttaccttccctccctcaatcggttgaatgtcgcccttttgtctttggcgctggtaaaccatatgaattttctattgattgtgacaaaataaacttattccgtggtgtctttgcgtttcttttatatgttgccacctttatgtatgtattttctacgtttgctaacatactgcgtaataaggagtcttaatcatgccagttcttttgggtattccgttattattgcgtttcctcggtttccttctggtaactttgttcggctatctgcttacttttcttaaaaagggcttcggtaagatagctattgctatttcattgtttcttgctcttattattgggcttaactcaattcttgtgggttatctctctgatattagcgctcaattaccctctgactttgttcagggtgttcagttaattctcccgtctaatgcgcttccctgtttttatgttattctctctgtaaaggctgctattttcatttttgacgttaaacaaaaaatcgtttcttatttggattgggataaataatatggctgtttattttgtaactggcaaattaggctctggaaagacgctcgttagcgttggtaagattcaggataaaattgtagctgggtgcaaaatagcaactaatcttgatttaaggcttcaaaacctcccgcaagtcgggaggttcgctaaaacgcctcgcgttcttagaataccggataagccttctatatctgatttgcttgctattgggcgcggtaatgattcctacgatgaaaataaaaacggcttgcttgttctcgatgagtgcggtacttggtttaatacccgttcttggaatgataaggaaagacagccgattattgattggtttctacatgctcgtaaattaggatgggatattatttttcttgttcaggacttatctattgttgataaacaggcgcgttctgcattagctgaacatgttgtttattgtcgtcgtctggacagaattactttaccttttgtcggtactttatattctcttattactggctcgaaaatgcctctgcctaaattacatgttggcgttgttaaatatggcgattctcaattaagccctactgttgagcgttggctttatactggtaagaatttgtataacgcatatgatactaaacaggctttttctagtaattatgattccggtgtttattcttatttaacgccttatttatcacacggtcggtatttcaaaccattaaatttaggtcagaagatgaaattaactaaaatatatttgaaaaagttttctcgcgttctttgtcttgcgattggatttgcatcagcatttacatatagttatataacccaacctaagccggaggttaaaaaggtagtctctcagacctatgattttgataaattcactattgactcttctcagcgtcttaatctaagctatcgctatgttttcaaggattctaagggaaaattaattaatagcgacgatttacagaagcaaggttattcactcacatatattgatttatgtactgtttccattaaaaaaggtaattcaaatgaaattgttaaatgtaattaattttgttttcttgatgtttgtttcatcatcttcttttgctcaggtaattgaaatgaataattcgcctctgcgcgattttgtaacttggtattcaaagcaatcaggcgaatccgttattgtttctcccgatgtaaaaggtactgttactgtatattcatctgacgttaaacctgaaaatctacgcaatttctttatttctgttttacgtgcaaataattttgatatggtaggttctaacccttccattattcagaagtataatccaaacaatcaggattatattgatgaattgccatcatctgataatcaggaatatgatgataattccgctccttctggtggtttctttgttccgcaaaatgataatgttactcaaacttttaaaattaataacgttcgggcaaaggatttaatacgagttgtcgaattgtttgtaaagtctaatacttctaaatcctcaaatgtattatctattgacggctctaatctattagttgttagtgctcctaaagatattttagataaccttcctcaattcctttcaactgttgatttgccaactgaccagatattgattgagggtttgatatttgaggttcagcaaggtgatgctttagatttttcatttgctgctggctctcagcgtggcactgttgcaggcggtgttaatactgaccgcctcacctctgttttatcttctgctggtggttcgttcggtatttttaatggcgatgttttagggctatcagttcgcgcattaaagactaatagccattcaaaaatattgtctgtgccacgtattcttacgctttcaggtcagaagggttctatctctgttggccagaatgttccttttattactggtcgtgtgactggtgaatctgccaatgtaaataatccatttcagacgattgagcgtcaaaatgtaggtatttccatgagcgtttttcctgttgcaatggctggcggtaatattgttctggatattaccagcaaggccgatagtttgagttcttctactcaggcaagtgatgttattactaatcaaagaagtattgctacaacggttaatttgcgtgatggacagactcttttactcggtggcctcactgattataaaaacacttctcaggattctggcgtaccgttcctgtctaaaatccctttaatcggcctcctgtttagctcccgctctgattctaacgaggaaagcacgttatacgtgctcgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgctttgcctggtttccggcaccagaagcggtgccggaaagctggctggagtgcgatcttcctgaggccgatactgtcgtcgtcccctcaaactggcagatgcacggttacgatgcgcccatctacaccaacgtgacctatcccattacggtcaatccgccgtttgttcccacggagaatccgacgggttgttactcgctcacatttaatgttgatgaaagctggctacaggaaggccagacgcgaattatttttgatggcgttcctattggttaaaaaatgagctgatttaacaaaaatttaatgcgaattttaacaaaatattaacgtttacaatttaaatatttgcttatacaatcttcctgtttttggggcttttctgattatcaaccggggtacatatgattgacatgctagttttacgattaccgttcatcgattctcttgtttgctccagactctcaggcaatgacctgatagcctttgtagatctctcaaaaatagctaccctctccggcattaatttatcagctagaacggttgaatatcatattgatggtgatttgactgtctccggcctttctcacccttttgaatctttacctacacattactcaggcattgcatttaaaatatatgagggttctaaaaatttttatccttgcgttgaaataaaggcttctcccgcaaaagtattacagggtcataatgtttttggtacaaccgatttagctttatgctctgaggctttattgcttaattttgctaattctttgccttgcctgtatgatttattggatgtt

SEQ ID NO: 10 - DNA, 41 bp, Artificial Sequence
  BamH I_SM_upper which is a primer used for site-directed mutation
aaggccgcttttgcgggatcctcaccctcagcagcgaaaga

SEQ ID NO: 11 - DNA, 41 bp, Artificial Sequence
  BamH I_SM_lower which is a primer used for site-directed mutation
tctttcgctgctgagggtgaggatcccgcaaaagcggcctt

SEQ ID NO: 12 - DNA, 90 bp, Artificial Sequence
  BamM13HK_P8_primer which is an extension primer used for preparation
ttaatggaaacttcctcatgaaaaagtctttagtcctcaaagcctctgtagccgttgctaccctcgttccgatgctgtctttcgctgctg

SEQ ID NO: 13 - DNA, 95 bp, Artificial Sequence
  M13HK_P8 which is a library oligonucleotide used for preparation
aaggccgcttttgcgggatccnnmnnmnnmnnmnnmnnmnnmncagcagcgaaagacagcatcggaacgagggtagcaacggctacagaggcttt

SEQ ID NO: 14 - protein, 50 aa, Artificial Sequence
  P8 protein of M13 phage
AEGDDPAKAAFNSLQASATEYIGYAWAMVVVIVGATIGIKLFKKFTSKAS